(19) United States
(12) Patent Application Publication
(10) Pub. No.: US 2021/0016150 A1
(43) Pub. Date: Jan. 21, 2021

(54) DEVICE AND METHOD FOR RECOGNIZING FREE WEIGHT TRAINING MOTION AND METHOD THEREOF

(71) Applicant: Jae Hoon Jeong, Seoul (KR)

(72) Inventors: Jae Hoon Jeong, Seoul (KR); Ha Yeon Park, Seoul (KR)

(21) Appl. No.: 16/931,360

(22) Filed: Jul. 16, 2020

(30) Foreign Application Priority Data
Jul. 17, 2019 (KR) 10-2019-0086480

(51) Int. Cl. A63B 71/06

(52) U.S. Cl. CPC A63B 71/0619; A63B 2220/806; A63B 2071/0638; A63B 2220/833; A63B 2071/0677; A63B 2220/802; A63B 2071/0647

(57) ABSTRACT

According to an embodiment, a device for recognizing a free weight training motion comprises a depth camera obtaining image information for measuring a user's body information before exercise and obtaining image information including a unique identifier attached to a piece of exercise equipment and motion of each joint of the user and a variation in body surface due to a motion of the body of the user doing exercise using the piece of exercise equipment, a sensor unit measuring a state of the piece of exercise equipment and recognizing the user, and a controller controlling the depth camera and the sensor unit and processing various pieces of information obtained by the depth camera and the sensor unit.

### CROSS-REFERENCE TO RELATED APPLICATION(S)

This application is based on and claims priority under 35 U.S.C. 119 to Korean Patent Application No. 10-2019-0086480, filed on Jul. 17, 2019, in the Korean Intellectual Property Office, the disclosure of which is herein incorporated by reference in its entirety.

### TECHNICAL FIELD

Embodiments of the disclosure relate to a device and method for recognizing or obtaining various pieces of information, including the type, repeat count, and weight of motion, including the phase, angle, speed, displacement, and depth of body joints of a user doing exercise using a piece of exercise equipment, via a depth camera or various sensors, and recognizing the free weight training motion being done by the user based on the recognized or obtained information.

### DESCRIPTION OF RELATED ART

Weight training is a resistance exercise to develop muscle strength and is divided into free weight training and machine weight training.

Exercise amount measuring devices for exercise equipment for free weight training are not widely used due to their high cost and complicated structure and are incapable of accurate measurement on various free weight training exercises.

If the user repeats free weight training in an incorrect position or without the weight normally supported, the user is put at risk of injury.

### SUMMARY

According to an embodiment, a device for recognizing a free weight training motion comprises a depth camera obtaining image information for measuring a user's body information before exercise and obtaining image information including a unique identifier attached to a piece of exercise equipment and motion of each joint of the user and a variation in body surface due to a motion of the body of the user doing exercise using the piece of exercise equipment, a sensor unit measuring a state of the piece of exercise equipment and recognizing the user, and a controller identifying motion information of a joint, on which the user is doing exercise using the piece of exercise equipment, type and repeat count of exercise, the user's body information, kind and weight of the piece of exercise equipment, and foot position and per-foot section pressure distribution, over time, upon exercise, based on the obtained image information and the measured state of piece of exercise equipment, identifying the free weight training motion being done by the user using the piece of exercise equipment, based on the identified joint motion information, exercise type and repeat count, the identified kind and weight of the piece of exercise equipment, and the identified foot position and per-foot section pressure distribution, comparing standard posture information according to exercise types pre-configured per piece of exercise equipment, previously stored in a storage unit, with the identified user body information, joint motion information, weight of the piece of exercise equipment according to the exercise type and repeat count, and foot position and per-foot section pressure distribution, and generating feedback information related to the user's free weight training motion according to a result of the comparison.

The sensor portion may include an ultrasonic sensor measuring a motion range of section in which the piece of exercise equipment is repeatedly moved, an radio frequency identification (RFID) reader recognizing the unique identifier attached to the piece of exercise equipment, a hall sensor measuring a magnitude of a magnetic field for a magnet attached to the piece of exercise equipment, a magnetic sensor measuring a magnitude of a magnetic field for a magnet attached to the piece of exercise equipment, a pressure plate measuring the user's body motion and foot pressure per exercise section for the motion of each joint, over time, for the user doing exercise using the piece of exercise equipment, a face recognition sensor recognizing the user doing exercise using the piece of exercise equipment, a fingerprint recognition sensor recognizing the user doing exercise using the piece of exercise equipment, and an iris recognition sensor recognizing the user doing exercise using the piece of exercise equipment.

The controller may identify the type and repeat count of exercise being done by the user using the piece of exercise equipment, based on at least one of the motion of each joint of the user in the obtained image information and the motion range of section, in which the piece of exercise equipment is repeatedly moved, the motion range of section being included in the measured state of the piece of exercise equipment, identify the kind and weight of the piece of exercise equipment corresponding to the unique identifier, based on at least one of the unique identifier attached to the piece of exercise equipment in the obtained image information and a unique identifier recognized from the piece of exercise equipment included in the measured state of the piece of exercise equipment, and identify at least one of the foot position and per-foot section pressure distribution, over time, upon exercise, based on the per-exercise section foot pressure for the motion of the body and each joint, over time, included in the measured state of the piece of exercise equipment.

According to an embodiment, a method for recognizing a free weight training motion comprises obtaining, by a depth camera, image information for measuring a user's body information before exercise and image information including a unique identifier attached to a piece of exercise equipment and motion of each joint of the user and a variation in body surface due to a motion of the body of the user doing exercise using the piece of exercise equipment, measuring, by a sensor unit, a state of the piece of exercise equipment and recognizing the user;

identifying, by a controller, joint motion information, type, and repeat count of exercise being done by the user using the piece of exercise equipment, based on at least one of the motion of each joint of the user in the obtained image information and the motion range of section, in which the piece of exercise equipment is repeatedly moved, the motion range of section being included in the measured state of the piece of exercise equipment, identifying, by the controller, the kind and weight of the piece of exercise equipment corresponding to the unique identifier, based on at least one of the unique identifier attached to the piece of exercise equipment in the obtained image information and a unique identifier recognized from the piece of exercise equipment included in the measured state of the piece of exercise equipment, identifying, by the controller, at least one of the foot position and per-foot section pressure distribution, over time, upon exercise, based on the per-exercise section foot pressure for the motion of the body and each joint, over time, included in the measured state of the piece of exercise equipment, identifying, by the controller, a free weight training motion being done by the user using the piece of exercise equipment, based on the identified joint motion information, exercise type and repeat count, the identified kind and weight of the piece of exercise equipment, and the identified foot position and per-foot section pressure distribution, comparing, by the controller, standard posture information according to exercise types pre-configured per piece of exercise equipment, previously stored in a storage unit, with the identified user body information, joint motion information, weight of the piece of exercise equipment according to the exercise type and repeat count, and foot position and per-foot section pressure distribution, generating, by the controller, feedback information related to the user's free weight training motion according to a result of the comparison, and providing, by the controller, the generated feedback information to the user.

The feedback information may include at least one of optimal exercise course information customized to the user, information as to whether the user's exercise goal is achieved, a dynamical element of each joint according to the user's exercise goal, information for partial strengthening exercise, information for reeducation of an exercise technique, comparison image information for the standard posture information and the captured motion of the user's body and each joint or foot position, a result of user recognition, and the user's body information.

According to an embodiment, various pieces of information, such as motion for, e.g., the phase, angle, speed, displacement, and depth of major body joints of the user doing exercise using the piece of exercise equipment, exercise type, exercise repeat count, and exercise weight may be recognized using the depth camera or various sensors, and the free weight training motion which the user is doing on the piece of exercise equipment may be recognized based on the recognized pieces of information. Thus, the free weight training motion being done by the user may be identified easily and conveniently.

According to an embodiment, feedback for the user's body information, reeducation of exercise technique, and partial reinforcing exercise may be provided via comparison between pre-stored pieces of information related to the free weight training motion corresponding to an athlete with superior performance and the optimal exercise course with pieces of information gathered in relation to the free weight training motion the user is doing using the piece of exercise equipment. Thus, the user may be given the optimized customized exercise type, and a desired part may be specifically reinforced.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a block diagram illustrating a configuration of a smart free weight training motion recognition device according to an embodiment of the disclosure;

FIG. 2 is a block diagram illustrating a configuration of a sensor unit according to an embodiment of the disclosure;

FIG. 3 is a view illustrating variations in foot pressure per lifting exercise interval according to an embodiment of the disclosure;

FIGS. 4, 5, 6, 7, 8, 9, 10, 11, 12, 13, 14, 15, 16, 17, 18, 19, 20, 21, 22, 23, 24, 25, 26, 27, and 28 are views illustrating various kinds of free weight training according to an embodiment of the disclosure;

FIGS. 29 and 30 are views illustrating an example of Q-angle according to an embodiment of the disclosure;

FIG. 31 is a view illustrating an example body median line according to an embodiment of the disclosure;

FIG. 32 is a view illustrating an example body median line and gravity line according to an embodiment of the disclosure; and

FIGS. 33 and 34 are flowcharts illustrating a smart free weight training motion recognition method according to an embodiment of the disclosure.

### DETAILED DESCRIPTION

The terms as used herein are provided merely to describe some embodiments thereof, but not to limit the present disclosure. The terms as used herein are provided merely to describe some embodiments thereof, but not to limit the scope of other embodiments of the present disclosure. Unless otherwise defined, all terms including technical and scientific terms used herein have the same meaning as commonly understood by one of ordinary skill in the art to which the embodiments of the present disclosure pertain and should not be interpreted as overly broad or narrow. As used herein, terms wrong or inappropriate for representing the spirit of the present disclosure may be replaced with and understood as more proper ones to represent the spirit of the present disclosure by one of ordinary skill in the art. General terms as used herein should be interpreted in the context of the specification or as defined in dictionaries.

As used herein, the singular forms “a,” “an,” and “the” are intended to include the plural forms as well, unless the context clearly indicates otherwise. As used herein, the term “comprise,” “include,” or “have” should be appreciated not to preclude the presence or addability of features, numbers, steps, operations, components, parts, or combinations thereof as set forth herein.

The terms coming with ordinal numbers such as ‘first’ and ‘second’ may be used to denote various components, but the components are not limited by the terms. The terms are used only to distinguish one component from another. For example, a first component may be denoted a second component, and vice versa without departing from the scope of the present disclosure.

Hereinafter, exemplary embodiments of the inventive concept will be described in detail with reference to the accompanying drawings. The same reference denotations may be used to refer to the same or substantially the same elements throughout the specification and the drawings. No duplicate description of the same elements is given herein. When determined to make the subject matter of the present disclosure unclear, the detailed description of the known art or functions may be skipped. The accompanying drawings are provided for an easier understanding of the spirit of the reception but the present disclosure should not be limited thereby.

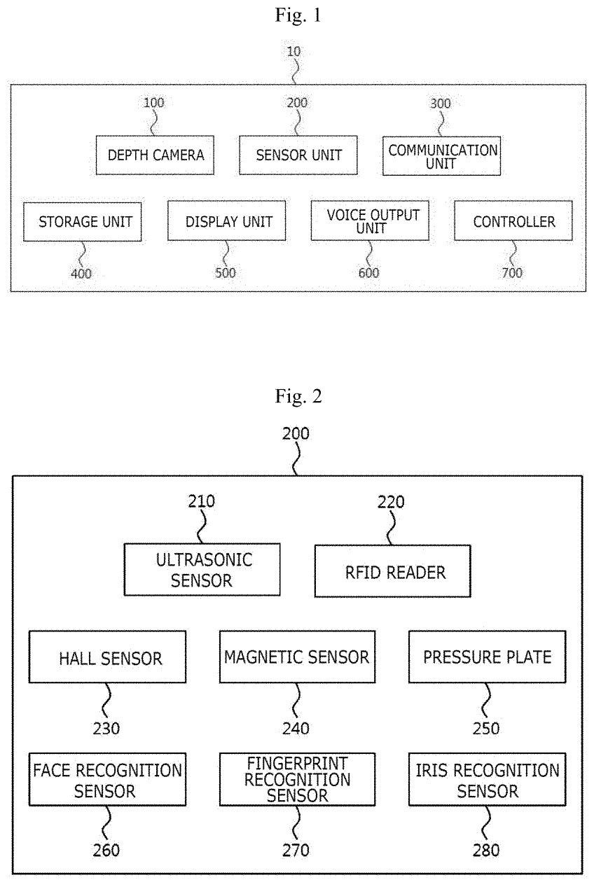

FIG. 1 is a block diagram illustrating a configuration of a smart free weight training motion recognition device according to an embodiment of the disclosure.

Referring to FIG. 1, a smart free weight training motion recognition device 10 includes a depth camera 100, a sensor unit 200, a communication unit 300, a storage unit 400, a display unit 500, a voice output unit 600, and a controller 700. All of the components of the smart free weight training motion recognition device 10 shown in FIG. 1 are not essential components, and the smart free weight training motion recognition device 10 may be implemented with more or less components than those shown in FIG. 1. The sensor unit 200, the communication unit 300, the storage unit 400, the display unit 500, and the voice output unit 600 may be implemented as, or may include, at least one circuit to implement or execute their respective functions. For example, the sensor unit 200 may be (or include) a sensor circuit. The communication unit 300 may be (or include) a communication circuit. The storage unit 400 may be (or include) a storage circuit. The display unit 500 may be (or include) a display circuit. The voice output unit 600 may be (or include) a voice output circuit.

According to an embodiment, the smart free weight training motion recognition device 10 may be applicable to various terminals or devices, such as smartphones, portable terminals, mobile terminals, foldable terminals, personal digital assistants (PDAs), portable multimedia players (PMPs), telematics terminals, navigation terminals, personal computers, laptop computers, slate PCs, tablet PCs, ultrabook computers, wearable devices, such as smartwatches, smart glasses, head-mounted displays, etc., Wibro terminals, Internet protocol television (IPTV) terminals, smart TVs, digital broadcast terminals, audio video navigation (AVN) terminals, audio/video (A/V) systems, flexible terminals, or digital signage devices.

Free weight training, a kind of weight training, refers to one's exercise using her own body (such as squats, lunges, chin-ups, or push-ups) or using barbells, dumbbells, or kettlebells, or E-Z bars and features a wide range of joint motion which allows free body exercises.

Machine weight training, another kind of weight training, refers to an exercise using a machine (or equipment) in training gyms, such as the leg press, chest press, or seated row and allows for a safe exercise due to a range of joint motion limited by the machine.

Accurate exercise methods, repetitions, and proper weight training provide humans with various benefits by developing the strength and muscles, but repeated incorrect motions during exercise or abnormal weight support may cause injuries.

In the case of weightlifting, for example, various muscles of the body are used during exercise. Accurate exercise technique, weight distribution of the feet for each exercise section, and exercise speed (or power) of the joints for each exercise section are critical factors to determine the performance.

According to the technical features of the present disclosure, it is possible to perform partial reinforcement or retraining of exercise techniques by comparison with the foot pressure distribution and exercise technique, per exercise section, of the athletes with excellent performance.

As used herein, “exercise section” means that the body uses a different muscle per exercise section. This means that the muscles of a desired portion may be specifically reinforced by per-exercise section measurement.

Free weight training exercises have some features as follows.

First, standing exercises on the ground feature that repeated motions occur only in a specific range (or height from the ground). Upon (or during) exercise, regarding the measurement about how high, or in what range, the repeated motions occur relative to the height (e.g., in the case of the shoulder press, 83% to 116%), the type of exercise and the number of repetitions of exercise may be known by measuring the height of the repeated motions from the ground, with an ultrasonic sensor mounted (or embedded or attached) on the dumbbell, barbell, or other piece of exercise equipment.

Second, as another measurement method, the type and number of repetitions of exercise, and the kind and weight of the piece of exercise equipment may be known by capturing, recording, or measuring the joints that are repeatedly moved, using a depth camera.

Third, the above-described two methods may be applied individually or in combination.

Fourth, an ideal exercise may be proposed by identifying the left/right/front/back weight distributions of the body and feet upon exercise, using the above-described methods.

According to an embodiment, the following measurement elements may be included as elements for measuring an exercise.

The measurement elements may include measurement of the type of exercise (e.g., determination as to what exercise is being done by recognizing the height and motions), the number of repetitions of exercise (e.g., measurement of the height in which the barbell or dumbbell is repeatedly moved or determination of what body joints are repeatedly moved), the weight of the exercise being done (e.g., measurement of the weight in kg of the barbell or dumbbell), measurement and analysis of the motion (including, e.g., the phase, angle, speed, or displacement of the joint motion) using the depth camera, measurement and analysis using per-motion section time information, measurement and analysis of the foot pressure for the body and joint motion using a pressure plate, measurement and analysis of the foot pressure distribution and per-foot section pressure using per-motion section temporal analysis, measurement and analysis of the foot pressure and body and joint motion using the depth camera and pressure plate, measurement and analysis of the foot pressure and motion using per-measurement section time information, and measurement of, e.g., the amount of exercise via joint motion.

At least one or more depth cameras 100 may be configured, provided, or placed on the front surface and/or side surface of the user who is doing exercise, using a piece of exercise equipment.

The depth camera 100 may be a three-dimensional (3D) depth camera and may be a kind of computational camera. The depth camera 100 may compute the depth value of each image pixel which two-dimensional (2D) cameras cannot.

Further, the depth camera 100 may emit a preconfigured light pattern and capture the distorted light pattern, thereby recognizing the 3D object or 3D structure.

For example, the depth camera 100 may compute the depth of the object based on the interval, deformation, or size of the captured light pattern.

The depth camera 100 may obtain or capture image information (or pre-exercise image information) including, e.g., the motion of the user's body and/or each joint before exercise.

The depth camera 100 may obtain (or capture) image information including, e.g., the motion of the user's body and/or each joint (or variations in the user's body surface due to the motion of the user's body and/or each joint during exercise) and the unique identifier (ID) (or unique image ID) attached the piece of equipment on which the user is doing exercise, on the front surface and/or side surface of the user on exercise. The variations in the user's body surface due to the motion of each joint include, e.g., the phase, angle, speed, displacement, and depth due to the joint motion. The piece of equipment includes at least one of barbells, dumbbells, kettlebells, or E-Z bars. The unique ID may include a difference piece of information depending on the kind or weight of the piece of equipment. The image information may be image information resultant from capturing the motion of the user's body and/or joint on the front surface and/or side surface of the user doing exercise on the piece of equipment. The depth camera 100 may obtain voice information around the user, as well as the image information.

According to an embodiment of the disclosure, a time-of-flight (ToF)-type camera is described herein as an example, but embodiments of the disclosure are not limited thereto. For example, a stereo-type or structured pattern depth camera may be used as the depth camera 100.

Or, other various types of depth cameras may be used which are able to recognize the shape and distance (or depth) of the object and detect a motion of the object.

The depth camera 100 may be a Kinect camera.

For example, the Kinect camera may include a red-green-blue (RGB) camera (not shown) for obtaining the color image (color view) of the object (or user) in front thereof, a 3D depth sensor (not shown) for extracting the depth information (or distance information) about the object and the image (depth view) indicating the depth information by emitting (or radiating) infrared light in pixel units to the object in front using an infrared (IR) emission projector (not shown) and an IR camera (not shown), and a multi-array microphone (MIC) for obtaining ambient sounds or voice information.

Other than using the Kinect camera, the depth camera 100 may adopt a motion capture scheme or fiber Bragg grating (FGB) scheme capable of recognizing (or computing/measuring the depth) the surface of the object (or 3D or stereoscopic object).

Other than using the Kinect camera, the depth camera 100 may use an RGB camera, IMU sensor, accelerometer, shape sensor, FGB, laser sensor, location sensor, Lidar sensor, multi-camera, optical marker, magnetic sensor, IR sensor, optical sensor, or semiconductor sensor to measure the surface (or depth) of the object.

Referring to FIG. 2, the sensor unit 200 may include an ultrasonic sensor 210, a radio frequency ID (RFID) reader 220, a hall sensor 230, a magnetic sensor 240, a pressure plate 250, a face recognition sensor 260, a fingerprint recognition sensor 270, and an iris recognition sensor 280. All of the components of the sensor unit 200 shown in FIG. 2 are not essential components, and the sensor unit 200 may be implemented with more or less components than those shown in FIG. 2.

The sensor unit 200 measures the state of the piece of exercise equipment on which the user is doing exercise and recognizes the user doing exercise on the piece of exercise equipment. The state of the piece of exercise equipment (or the state information about the piece of exercise equipment) may include information about the variation in height due to the time variation in the piece of exercise equipment over time as the piece of exercise equipment is used by the exercising user, the unique identifier of the piece of exercise equipment, magnitude of magnetic field, and per-exercise section foot pressure.

The ultrasonic sensor 210 measures the height of the section in which the piece of exercise equipment is repeatedly moved by the exercising user (or the range of motion of the section of motion). The ultrasonic sensor 210 may also measure variations in the height of the section in which the piece of exercise equipment is repeatedly moved over time.

The RFID reader 220 recognizes the unique ID attached to the piece of exercise equipment on which the user is doing exercise. The piece of exercise equipment may be in the state of different unique IDs (or tags including different unique IDs) attached depending on weights and types. The tags including different unique IDs may be attached to, or detached from, the piece of exercise equipment.

Where the piece of exercise equipment on which the user is doing exercise is a barbell, dumbbell, or E-Z bar which has (fixed) weights attached at the left and right, the RFID reader 220 may recognize each of the unique IDs in the tags individually attached to the left and right weights or recognizes the unique ID in one tag attached to one of the left and right weights. If the RFID reader 220 individually recognizes the unique IDs from the tags attached to the left and right weights, the weight of the piece of exercise equipment may be calculated by summating the weight information included in the recognized unique IDs (e.g., weight information about each of the left and right weights). If the RFID reader 220 recognizes the unique ID from one tag attached to one of the left and right weights, the weight of the piece of exercise equipment may be obtained based on the weight information (e.g., the weight information obtained by summating the left and right weights) included in the unique ID recognized from the tag.

The hall sensor 230 measures the magnitude of the magnetic field for the magnet attached to the piece of exercise equipment on which the user is doing exercise. In this case, the magnet which produce a different magnitude of magnetic field per weight may be attached to one side of the piece of exercise equipment.

The magnetic sensor 240 measures the magnitude of the magnetic field for the magnet attached to the piece of exercise equipment on which the user is doing exercise.

The hall sensor 230 or magnetic sensor 240 and the magnet attached to the piece of exercise equipment may be positioned at a radius within a preset error range so that the hall sensor 230 or magnetic sensor 240 may constantly measure the magnitude of magnetic field from the magnet attached to the piece of exercise equipment, thereby increasing accuracy.

The pressure plate (or pressure foot step) 250 measures the per-exercise section foot pressure for the motion of the body and/or each joint, over time, for the user doing exercise on the piece of exercise equipment. The exercise section refers to the state in which for a series of exercises the user is doing, specific muscles or joint angles used per step differ.

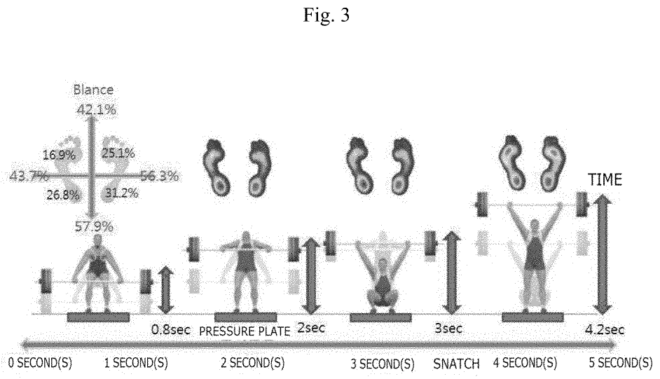

As shown in FIG. 3, if the user does exercise on the piece of exercise equipment in the state of stepping on the pressure plate 250, the pressure applied to the user's feet per exercise section is varied over time.

For example, as shown in FIG. 3, for the first and second sections (or motions) of the four sections (or motions) of the snatch (weightlifting), the user's foot pressure, joint angle, and exercise speed are compared with those of an Olympic gold medalist to thereby obtain relative comparison results for asymmetric and joint angle or speed, and the obtained comparison results may be used as information for the user's partial strengthening exercise. Such snatch motions may be used for symmetric comparison between left and right joints based on the per-exercise section foot pressure, per-section consumed time, median line, and gravity line (or center line) and to compute power by per-section exercise time measurement.

The face recognition sensor 260 is configured, provided, or placed on a side of the piece of exercise equipment, a side of the front surface of the user doing exercise on the piece of exercise equipment, or a side of the smart free weight training motion recognition device 10.

The face recognition sensor 260 recognizes the user doing exercise on the piece of exercise equipment (e.g., by Face ID).

The fingerprint recognition sensor 270 is configured, provided, or placed on a side of the piece of exercise equipment or a side of the smart free weight training motion recognition device 10.

The fingerprint recognition sensor 270 recognizes the user doing exercise on the piece of exercise equipment.

The iris recognition sensor 280 is configured, provided, or placed on a side of the piece of exercise equipment, a side of the front surface of the user doing exercise on the piece of exercise equipment, or a side of the smart free weight training motion recognition device 10.

The iris recognition sensor 280 recognizes the user doing exercise on the piece of exercise equipment.

The smart free weight training motion recognition device 10 may recognize the user based on the user's body information features (information, e.g., the lengths of the arm and leg and the pelvis-to-shoulder ratio) included in the image information captured by the depth camera 100.

As such, the depth camera 100 and the sensor unit 200 may be configured, provided, or placed on the front surface and/or side surface of the user doing exercise on the piece of exercise equipment, gathering (or obtaining or measuring) various pieces of information related to the exercising user on the user's front surface and/or side surface.

As compared with other animals, human motions in many cases occur in their standing position, influenced by gravity (which steadily applies vertical loads to the spinal muscles). Thus, if joint exercise is measured in the seated position or with the influence by gravity minimized, the joint measurement may not be said to be precise.

Measurement or prediction of exercise or motions, with the influence by gravity excluded, is an unscientific measurement scheme.

Thus, precise measurement of body muscles is a critical factor for predicting the athletic ability (or performance). The measurement method via the sensor unit 200, which measures the center of body weight using, e.g., the pressure plate 250, according to an embodiment of the disclosure may minimize abnormal stress upon exercise and thus absolutely affect the athletic ability and may aid in enhancing the user's athletic ability by providing a measurement scheme using the median line and gravity line (or center line) on the front/side surface of the body and the foot pressure.

The communication unit 300 forms a communication connection with any internal component or at least one external terminal via a wired/wireless communication network. The terminal may include a terminal (not shown) or a server (not shown). Wireless Internet technology which may be adopted for transmission over the wireless communication network may include at least one of wireless LAN (WLAN), digital living network alliance (DLNA), wireless broadband (Wibro), world interoperability for microwave access (Wimax), high speed downlink packet access (HSDPA), high speed uplink packet access (HSDPA), IEEE 802.16, long-term evolution (LTE), LTE-advanced (LTE-A), LTE V2X (C′V2X), wireless mobile broadband service (WMBS), or 5G communication. The communication unit 300 may transmit or receive data according to at least one wireless Internet technology which may include not only the above-enumerated techniques but also other non-mentioned Internet techniques. Short-range communication techniques that may be adopted according to an embodiment may include Bluetooth, radio frequency identification (RFID), infrared data association (IrDA), ultra-wideband (UWB), ZigBee, near-field communication (NFC), ultrasound communication (USC), visible light communication (VLC), Wi-Fi, or Wi-Fi direct. Wired communication techniques that may be adopted according to an embodiment may include power line communication (PLC), universal serial bus (USB) communication, Ethernet, serial communication, or optical/coaxial cable communication.

The communication unit 300 may communicate information or data with any terminal via a USB.

The communication unit 300 transmits or receives wireless signals to/from at least one of a base station, the terminal, or the server over a mobile communication network established as per mobile communication standards, protocols, or schemes, such as global system for mobile communication (GSM), code division multiple access (CDMA), CDMA2000, enhanced voice-data optimized or enhanced voice-data only (EV-DO), wideband CDMA (WCDMA), high speed downlink packet access (HSDPA), high speed uplink packet access (HSUPA), long term evolution (LTE), or LTE-advanced (LTE-A).

The communication unit 300 gathers various pieces of information measured by the sensor unit 200.

The storage unit 400 stores various user interfaces (UIs) and graphic user interfaces (GUIs).

The storage unit 400 stores a program and data necessary for the smart free weight training motion recognition device 10 to operate.

For example, the storage unit 400 may store a plurality of application programs (or simply applications) which may run on the smart free weight training motion recognition device 10 and data and instructions or commands for operations of the smart free weight training motion recognition device 10. At least some of the application programs may be downloaded from an external server via wireless communication. At least some of the application programs may be installed on the smart free weight training motion recognition device 10 before the smart free weight training motion recognition device 10 comes out on the market so as to be able to perform basic or default functions of the smart free weight training motion recognition device 10. The application programs may be stored in the storage unit 400 and be installed on the smart free weight training motion recognition device 10 and may be driven to perform operations (or functions) of the smart free weight training motion recognition device 10 by the controller 700.

The storage unit 400 may include at least one type of storage medium of flash memory types, hard disk types, multimedia card micro types, card types of memories (e.g., SD or XD memory cards), RAMs (Random Access Memories), SRAMs (Static Random Access Memories), ROMs (Read-Only Memories), EEPROMs (Electrically Erasable Programmable Read-Only Memories), PROMs (Programmable Read-Only Memories), magnetic memories, magnetic disks, or optical discs. The smart free weight training motion recognition device 10 may operate web storage which performs the storage function of the storage unit 400 over the Internet or may operate in association with the web storage.

The storage unit 400 stores various pieces of information measured by the sensor unit 200 and gathered under the control of the controller 700.

The storage unit 400 stores personal (or private) information related to any user. The personal information includes the user's name (or nickname), age, height, weight, and foot size.

The storage unit 400 stores (or manages) the ratio of body motion range to height per exercise type, information about the kind and weight of piece of exercise equipment per unique ID, information about the magnitude per weight of piece of exercise equipment, and standard posture information according to the exercise type preset per piece of exercise equipment.

In this case, the exercise type includes a barbell deadlift, a bent over barbell row, a barbell shoulder press, a barbell arm curl, a barbell bench press, an incline/decline barbell bench press, a barbell squat, a barbell lunge, a barbell shrug, a barbell upright row, and E-Z bar triceps extension, which use a barbell, a dumbbell deadlift, dumbbell row, dumbbell shoulder press, alternate dumbbell arm curl, dumbbell bench (or chest) press, Incline/decline dumbbell chest press, dumbbell squat, dumbbell lunges, dumbbell shrugs, dumbbell lateral rises, one arm dumbbell triceps extensions, and dumbbell kickbacks, which use dumbbells. The deadlift, row, shoulder press, arm curl, bench press, incline/decline bench press, squat, lunge, and shrug may be in different states of exercise equipment (e.g., barbell or dumbbell) used for the same exercise motion. Thus, the piece of exercise equipment may be identified via the unique ID recognized from the piece of exercise equipment or image analysis for the images in the image information.

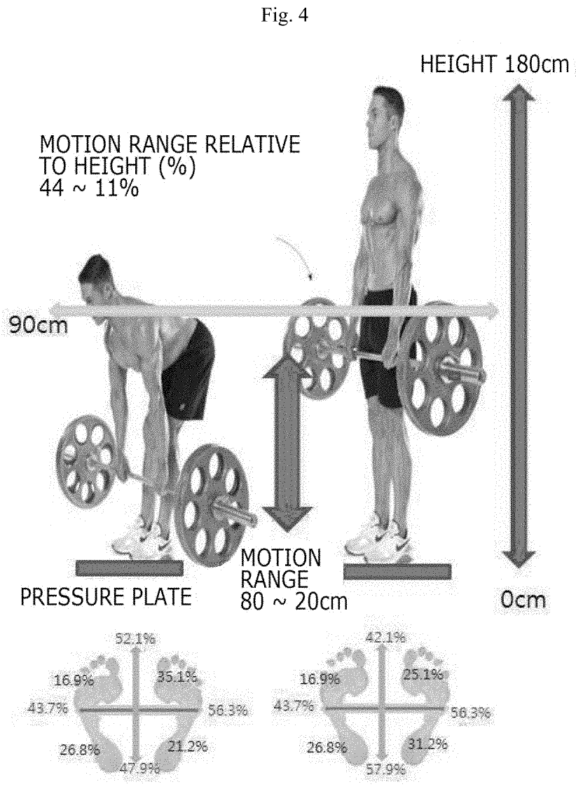

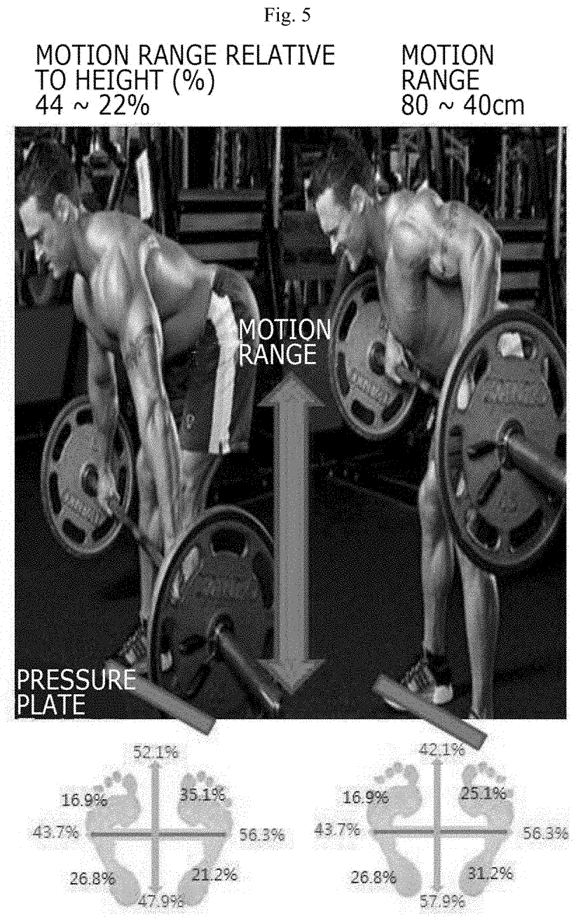

As shown in FIG. 4, the barbell deadlift is an exercise in which the barbell with the ultrasonic sensor 210 attached thereto is moved from the lower pelvis to the middle of the shin. For the barbell deadlift, the hip joints and shoulder joints are used, and lower leg back muscles are used. In the case where the identification is performed using the depth camera 100, upon free weight training by the barbell deadlift, only the hip joints are moved in the standing position and motion to overhead occurs. The identification may be performed in the range of the moving height by the depth camera 100 or ultrasonic sensor 210. For example, if the user is 180 cm tall, the motion ranges from about 80 cm to about 20 cm, and the ratio of body motion to height ranges from about 44% to about 11%.

As shown in FIG. 5, the bent over barbell row is an exercise in which the barbell with the ultrasonic sensor 210 attached thereto is moved from the lower pelvis to under the knee. For the bent over barbell row, the shoulder and elbow joints are used, and the gastrocnemius, biceps brachii, humerus radial muscle are used. In the case where the identification is performed using the depth camera 100, upon free weight training of bent over barbell row, only the upper arm (or extension exercise) and elbow (or flexion exercise) joints move, with the head lowered in a fixed position. The identification may be performed in the range of the moving height by the depth camera 100 or ultrasonic sensor 210. For example, if the user is 180 cm tall, the motion ranges from about 80 cm to about 40 cm, and the ratio of body motion to height ranges from about 44% to about 22%.

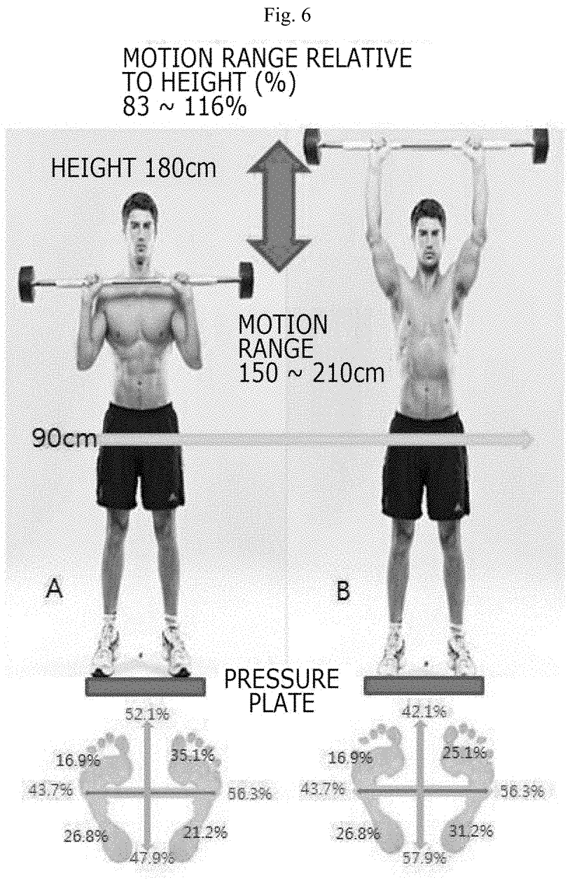

As shown in FIG. 6, the barbell shoulder press is an exercise in which the barbell with the ultrasonic sensor 210 attached thereto is moved from the clavicle to overhead. For the barbell shoulder press, the shoulder and elbow joints are used, and the deltoid muscles and musculus triceps brachii are used. In the case where the identification is performed using the depth camera 100, upon free weight training of the barbell shoulder press, only the shoulder joints (abduction exercise) and elbow joints (extension exercise) are moved in the standing position. The identification may be performed in the range of the moving height by the depth camera 100 or ultrasonic sensor 210. For example, if the user is 180 cm tall, the motion ranges from about 150 cm to about 210 cm, and the ratio of body motion to height ranges from about 83% to about 116%.

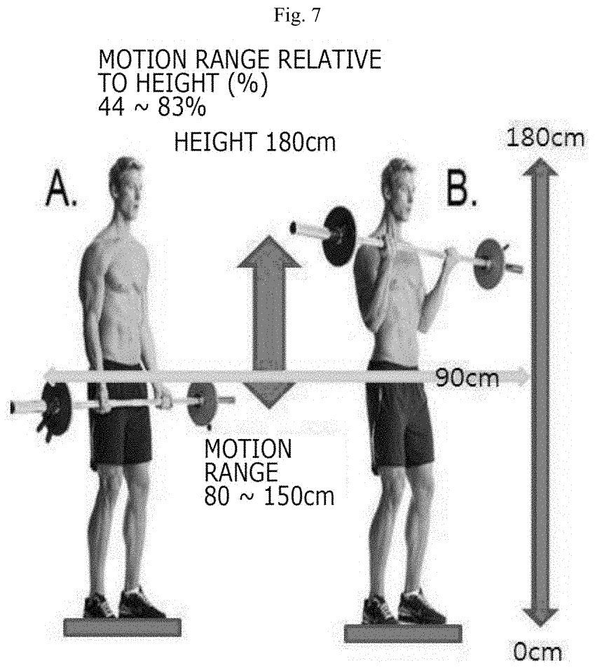

As shown in FIG. 7, the barbell arm curl is an exercise in which the barbell with the ultrasonic sensor 210 attached thereto is moved from the pelvis to the clavicle. For the barbell arm curl, the elbow joints and muscle biceps brachii are used. In the case where the identification is performed using the depth camera 100, upon free weight training of the barbell arm curl, only the elbow joints (flexion exercise) are moved in the standing position. The identification may be performed in the range of the moving height by the depth camera 100 or ultrasonic sensor 210. For example, if the user is 180 cm tall, the motion ranges from about 80 cm to about 150 cm, and the ratio of body motion to height ranges from about 44% to about 83%.

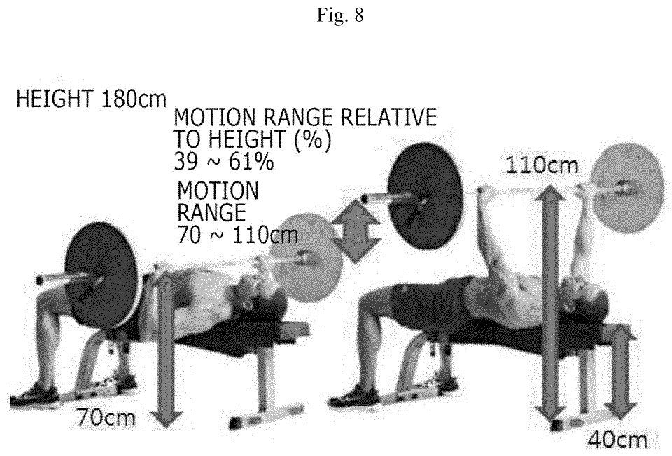

As shown in FIG. 8, the barbell bench press is an exercise in which the barbell with the ultrasonic sensor 210 attached thereto is moved from the clavicle to overhead. For the barbell shoulder press, the shoulder and elbow joints are used, and the pectoralis major and musculus triceps brachii are used. In the case where the identification is performed using the depth camera 100, upon free weight training of the barbell bench press, only the shoulders and elbows are moved in the lying position, with the knees bent and the head and pelvis level. The barbell bench press differs from the push-up in that it is performed with knees bent in the lying position. The identification may be performed in the range of the moving height by the depth camera 100 or ultrasonic sensor 210. For example, if the user is 180 cm tall, the motion ranges from about 70 cm to about 110 cm, and the ratio of body motion to height ranges from about 39% to about 61%.

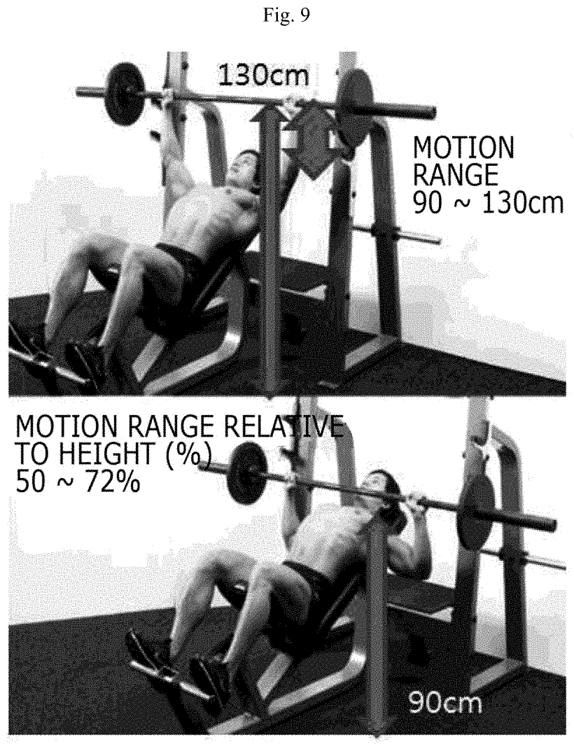

As shown in FIG. 9, the incline barbell bench press is an exercise in which the barbell with the ultrasonic sensor 210 attached thereto is moved from the clavicle to overhead. For the incline barbell bench press, the shoulder and elbow joints are used, and the pectoralis major and musculus triceps brachii are used. In the case where the identification is performed using the depth camera 100, upon free weight training of the incline barbell bench press, only the shoulders and elbows are moved in a side-lying position (in which the head is positioned higher than the pelvis), with the knees bent. The identification may be performed in the range of the moving height by the depth camera 100 or ultrasonic sensor 210. For example, if the user is 180 cm tall, the motion ranges from about 90 cm to about 130 cm, and the ratio of body motion to height ranges from about 50% to about 72%.

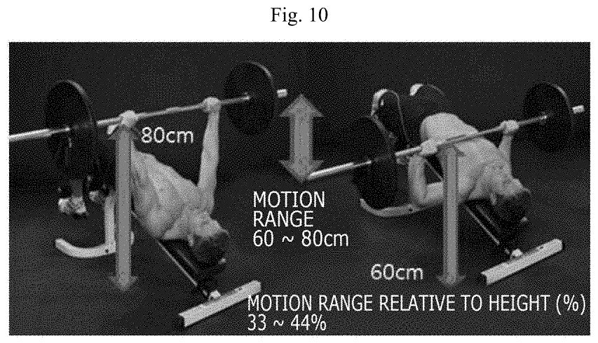

As shown in FIG. 10, the decline barbell bench press is an exercise in which the barbell with the ultrasonic sensor 210 attached thereto is moved from the clavicle to overhead. For the decline barbell bench press, the shoulder and elbow joints are used, and the pectoralis major and musculus triceps brachii are used. In the case where the identification is performed using the depth camera 100, upon free weight training of the decline barbell bench press, only the shoulders and elbows are moved in a side-lying position (in which the head is positioned lower than the pelvis), with the knees bent. The identification may be performed in the range of the moving height by the depth camera 100 or ultrasonic sensor 210. For example, if the user is 180 cm tall, the motion ranges from about 60 cm to about 80 cm, and the ratio of body motion to height ranges from about 33% to about 44%.

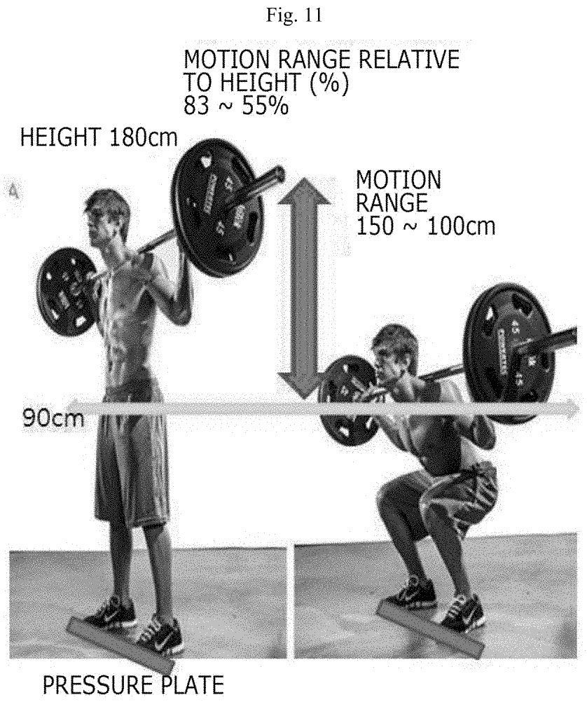

As shown in FIG. 11, the barbell squat is an exercise in which the barbell with the ultrasonic sensor 210 attached thereto is moved from the clavicle to belly button. For the barbell squat, the hip joints and knee joints are used, and the quadriceps, biceps femoris, semitendinosus, semimembranosus, and gluteus maximus are used. In the case where the identification is performed using the depth camera 100, upon free weight training of the barbell squat, both knees and hip joints simultaneously repeat flexion and extension in the standing position. The identification may be performed in the range of the moving height by the depth camera 100 or ultrasonic sensor 210. For example, if the user is 180 cm tall, the motion ranges from about 150 cm to about 100 cm, and the ratio of body motion to height ranges from about 83% to about 55%.

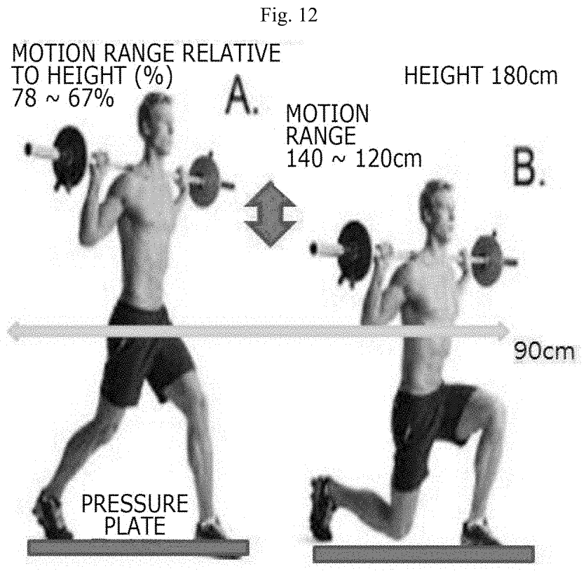

As shown in FIG. 12, the barbell lunge is an exercise in which the barbell with the ultrasonic sensor 210 attached thereto is moved from the clavicle to under the breast. For the barbell lunge, the hip joints and knee joints are used, and the quadriceps, biceps femoris, semitendinosus, semimembranosus, and gluteus maximus are used. In the case where the identification is performed using the depth camera 100, upon free weight training of the barbell lunge, the hip joint and knee joint for one leg positioned behind in the standing position, and only the knee joint for the other leg positioned ahead, repeat flexion and extension. The identification may be performed in the range of the moving height by the depth camera 100 or ultrasonic sensor 210. For example, if the user is 180 cm tall, the motion ranges from about 140 cm to about 120 cm, and the ratio of body motion to height ranges from about 78% to about 67%.

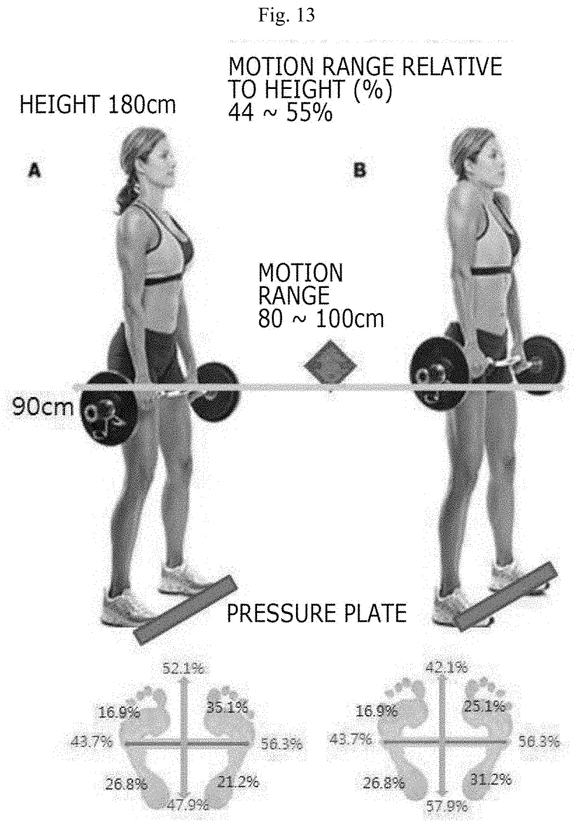

As shown in FIG. 13, the barbell shrug is an exercise in which the barbell with the ultrasonic sensor 210 attached thereto is moved from the trochanter major to a side of the pelvis. For the barbell shrug, the scapulothoracic joint and trapezii are used. In the case where the identification is performed using the depth camera 100, upon free weight training by the barbell shrug, only the height of the shoulders is varied in the standing position. The identification may be performed in the range of the moving height by the depth camera 100 or ultrasonic sensor 210. For example, if the user is 180 cm tall, the motion ranges from about 80 cm to about 100 cm, and the ratio of body motion to height ranges from about 44% to about 55%.

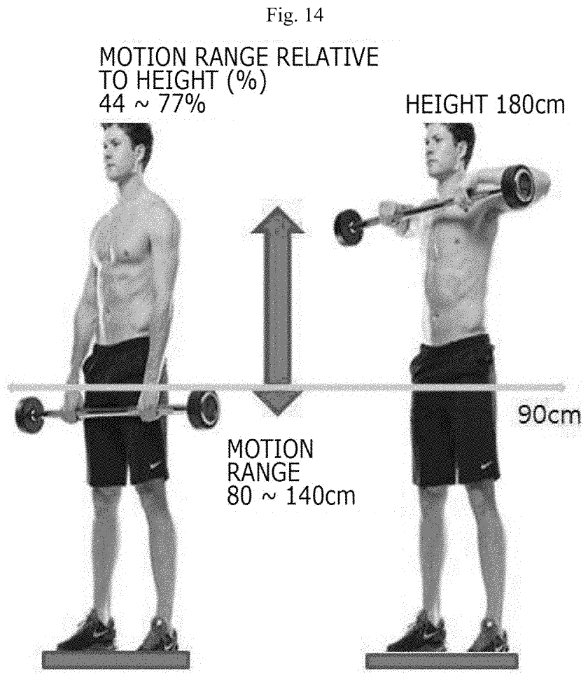

As shown in FIG. 14, the barbell upright row is an exercise in which the barbell with the ultrasonic sensor 210 attached thereto is moved from the belly button to the clavicle. For the barbell upright row, the shoulder and elbow joints and trapezii, deltoid, and brachioradialis are used. In the case where the identification is performed using the depth camera 100, upon free weight training of the barbell upright row, both shoulders and elbows are simultaneously moved, with the height of the elbows similar to the height of the shoulders in the standing position. The identification may be performed in the range of the moving height by the depth camera 100 or ultrasonic sensor 210. For example, if the user is 180 cm tall, the motion ranges from about 80 cm to about 140 cm, and the ratio of body motion to height ranges from about 44% to about 77%.

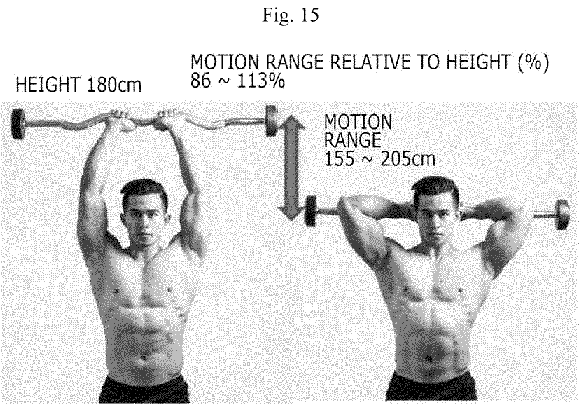

As shown in FIG. 15, the E-Z bar triceps extension is an exercise in which the E-Z bar with the ultrasonic sensor 210 attached thereto is moved from the center of the back of the head to overhead. For the E-Z bar triceps extension, the shoulder and elbow joints and musculus triceps brachii are used. In the case where the identification is performed using the depth camera 100, upon free weight training of the E-Z bar triceps extension, both arms are lifted in the standing position and, then, only the height of hands is varied only with an exercise on the elbow joints. The identification may be performed in the range of the moving height by the depth camera 100 or ultrasonic sensor 210. For example, if the user is 180 cm tall, the motion ranges from about 155 cm to about 205 cm, and the ratio of body motion to height ranges from about 86% to about 113%.

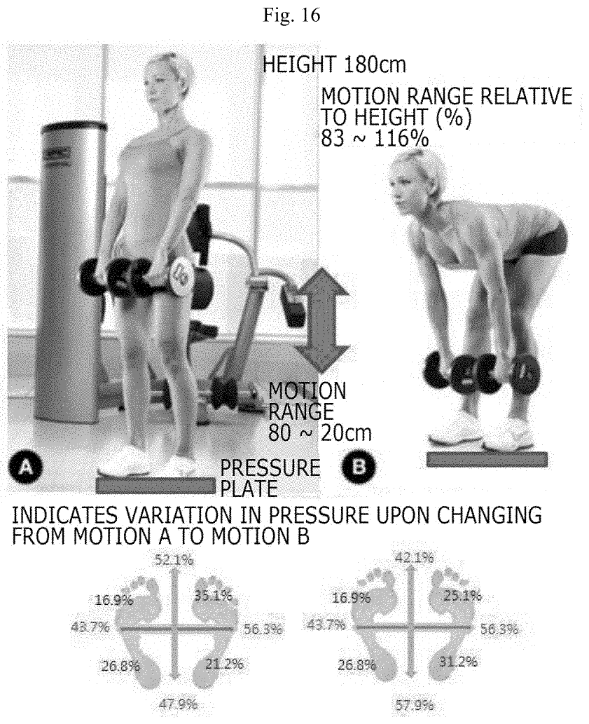

As shown in FIG. 16, the dumbbell deadlift is an exercise in which the dumbbell with the ultrasonic sensor 210 attached thereto is moved from the lower pelvis to the middle of the shin. For the dumbbell deadlift, the hip joints and shoulder joints are used, and lower leg back muscles are used. In the case where the identification is performed using the depth camera 100, upon free weight training of the dumbbell deadlift, only the hip joints are moved in the standing position and motion to overhead occurs. The identification may be performed in the range of the moving height by the depth camera 100 or ultrasonic sensor 210. For example, if the user is 180 cm tall, the motion ranges from about 80 cm to about 20 cm, and the ratio of body motion to height ranges from about 44% to about 11%.

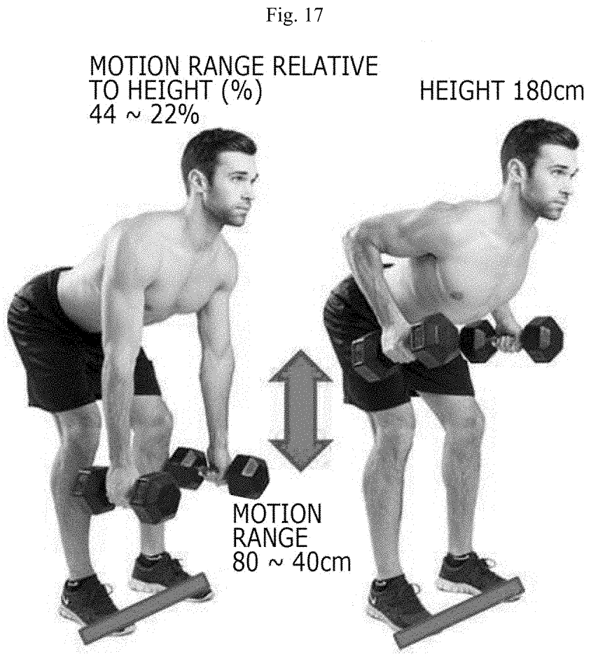

As shown in FIG. 17, the bent over dumbbell row is an exercise in which the dumbbell with the ultrasonic sensor 210 attached thereto is moved from the lower pelvis to under the knee. For the bent over dumbbell row, the shoulder and elbow joints are used, and the gastrocnemius, biceps brachii, humerus radial muscle are used. In the case where the identification is performed using the depth camera 100, upon free weight training of bent over dumbbell row, only the upper arm (or extension exercise) and elbow (or flexion exercise) joints move, with the head lowered in a fixed position. The identification may be performed in the range of the moving height by the depth camera 100 or ultrasonic sensor 210. For example, if the user is 180 cm tall, the motion ranges from about 80 cm to about 40 cm, and the ratio of body motion to height ranges from about 44% to about 22%.

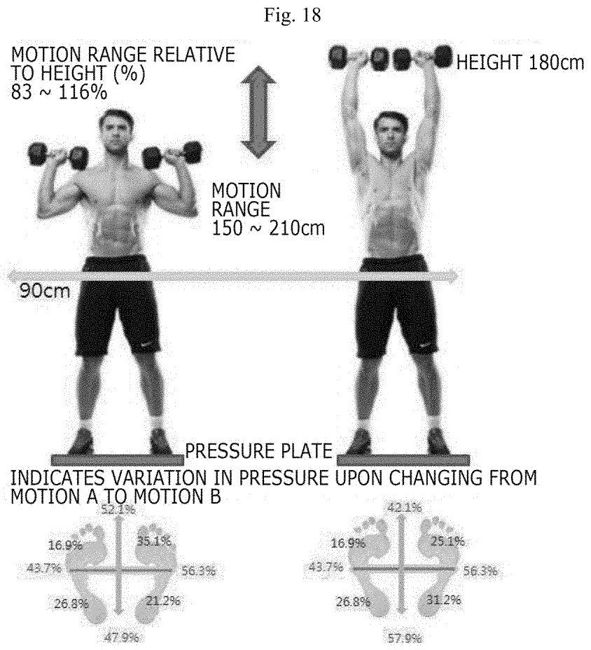

As shown in FIG. 18, the dumbbell shoulder press is an exercise in which the dumbbell with the ultrasonic sensor 210 attached thereto is moved from the clavicle to overhead. For the dumbbell shoulder press, the shoulder and elbow joints are used, and the deltoid muscles and musculus triceps brachii are used. In the case where the identification is performed using the depth camera 100, upon free weight training of the dumbbell shoulder press, only the shoulder joints (abduction exercise) and elbow joints (extension exercise) are moved in the standing position. The identification may be performed in the range of the moving height by the depth camera 100 or ultrasonic sensor 210. For example, if the user is 180 cm tall, the motion ranges from about 150 cm to about 210 cm, and the ratio of body motion to height ranges from about 83% to about 116%.

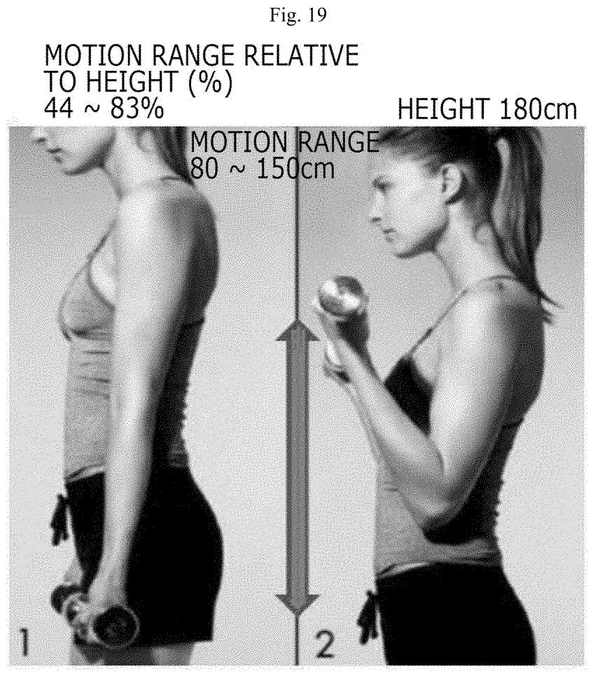

As shown in FIG. 19, the alternate dumbbell arm curl is an exercise in which the dumbbell with the ultrasonic sensor 210 attached thereto is moved from the pelvis to the clavicle. For the alternate dumbbell arm curl, the elbow joints and muscle biceps brachii are used. In the case where the identification is performed using the depth camera 100, upon free weight training of the alternate dumbbell arm curl, only the elbow joints (flexion exercise) are moved in the standing position. The identification may be performed in the range of the moving height by the depth camera 100 or ultrasonic sensor 210. For example, if the user is 180 cm tall, the motion ranges from about 80 cm to about 150 cm, and the ratio of body motion to height ranges from about 44% to about 83%.

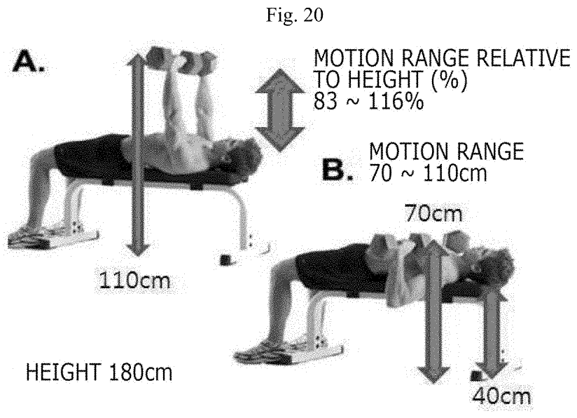

As shown in FIG. 20, the dumbbell bench press (or chest press) is an exercise in which the dumbbell with the ultrasonic sensor 210 attached thereto is moved from the clavicle to overhead. For the dumbbell shoulder press, the shoulder and elbow joints are used, and the pectoralis major and musculus triceps brachii are used. In the case where the identification is performed using the depth camera 100, upon free weight training of the dumbbell bench press, only the shoulders and elbows are moved in the lying position, with the knees bent and the head and pelvis level. The dumbbell bench press differs from the push-up in that it is performed with knees bent in the lying position. The identification may be performed in the range of the moving height by the depth camera 100 or ultrasonic sensor 210. For example, if the user is 180 cm tall, the motion ranges from about 70 cm to about 110 cm, and the ratio of body motion to height ranges from about 39% to about 61%.

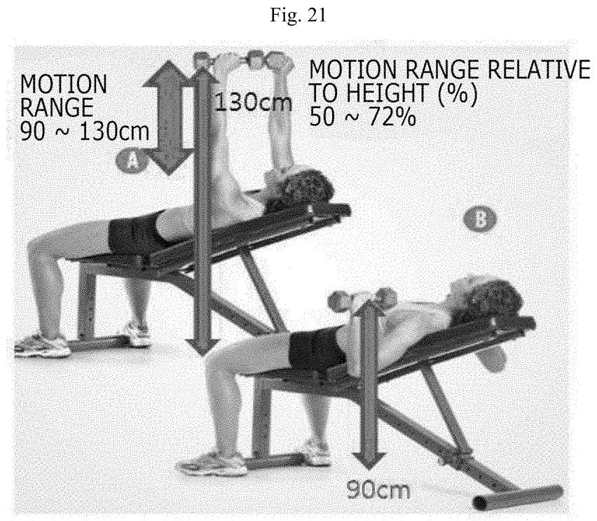

As shown in FIG. 21, the incline dumbbell chest press is an exercise in which the dumbbell with the ultrasonic sensor 210 attached thereto is moved from the clavicle to overhead. For the incline dumbbell chest press, the shoulder and elbow joints are used, and the pectoralis major and musculus triceps brachii are used. In the case where the identification is performed using the depth camera 100, upon free weight training of the incline dumbbell chest press, only the shoulders and elbows are moved in a side-lying position (in which the head is positioned higher than the pelvis), with the knees bent. The identification may be performed in the range of the moving height by the depth camera 100 or ultrasonic sensor 210. For example, if the user is 180 cm tall, the motion ranges from about 90 cm to about 130 cm, and the ratio of body motion to height ranges from about 50% to about 72%.

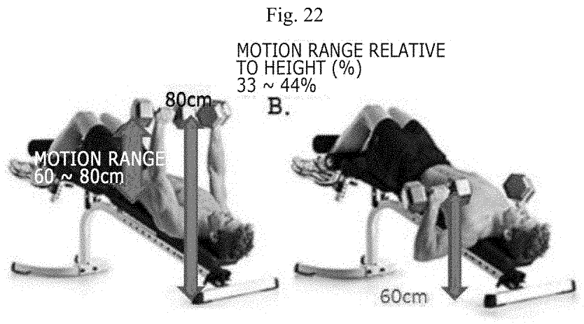

As shown in FIG. 22, the decline dumbbell chest press is an exercise in which the dumbbell with the ultrasonic sensor 210 attached thereto is moved from the clavicle to overhead. For the decline dumbbell chest press, the shoulder and elbow joints are used, and the pectoralis major and musculus triceps brachii are used. In the case where the identification is performed using the depth camera 100, upon free weight training of the decline dumbbell chest press, only the shoulders and elbows are moved in a side-lying position (in which the head is positioned lower than the pelvis), with the knees bent. The identification may be performed in the range of the moving height by the depth camera 100 or ultrasonic sensor 210. For example, if the user is 180 cm tall, the motion ranges from about 60 cm to about 80 cm, and the ratio of body motion to height ranges from about 33% to about 44%.

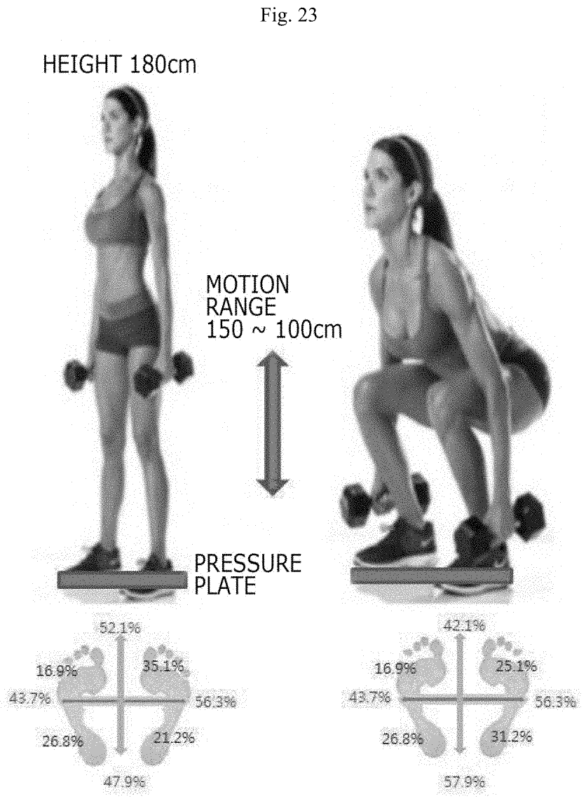

As shown in FIG. 23, the dumbbell squat is an exercise in which the dumbbell with the ultrasonic sensor 210 attached thereto is moved from the clavicle to belly button. For the dumbbell squat, the hip joints and knee joints are used, and the quadriceps, biceps femoris, semitendinosus, semimembranosus, and gluteus maximus are used. In the case where the identification is performed using the depth camera 100, upon free weight training of the dumbbell squat, both knees and hip joints simultaneously repeat flexion and extension in the standing position. The identification may be performed in the range of the moving height by the depth camera 100 or ultrasonic sensor 210. For example, if the user is 180 cm tall, the motion ranges from about 150 cm to about 100 cm, and the ratio of body motion to height ranges from about 83% to about 55%.

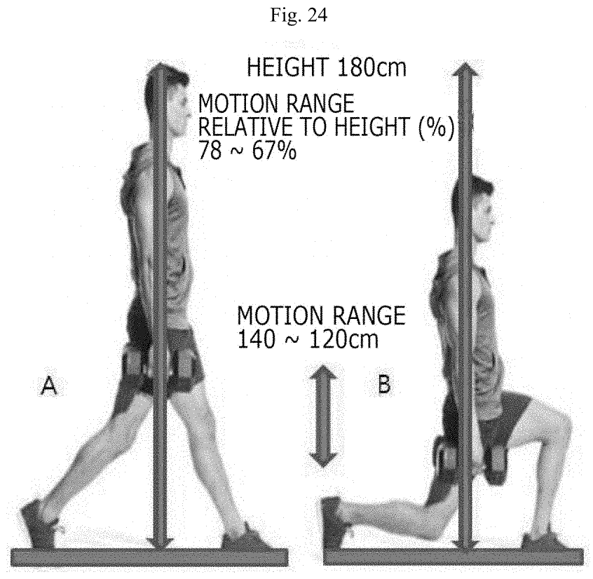

As shown in FIG. 24, the dumbbell lunge is an exercise in which the dumbbell with the ultrasonic sensor 210 attached thereto is moved from the clavicle to under the breast. For the dumbbell lunge, the hip joints and knee joints are used, and the quadriceps, biceps femoris, semitendinosus, semimembranosus, and gluteus maximus are used. In the case where the identification is performed using the depth camera 100, upon free weight training of the dumbbell lunge, the hip joint and knee joint for one leg positioned behind in the standing position, and only the knee joint for the other leg positioned ahead, repeat flexion and extension. The identification may be performed in the range of the moving height by the depth camera 100 or ultrasonic sensor 210. For example, if the user is 180 cm tall, the motion ranges from about 140 cm to about 120 cm, and the ratio of body motion to height ranges from about 78% to about 67%.

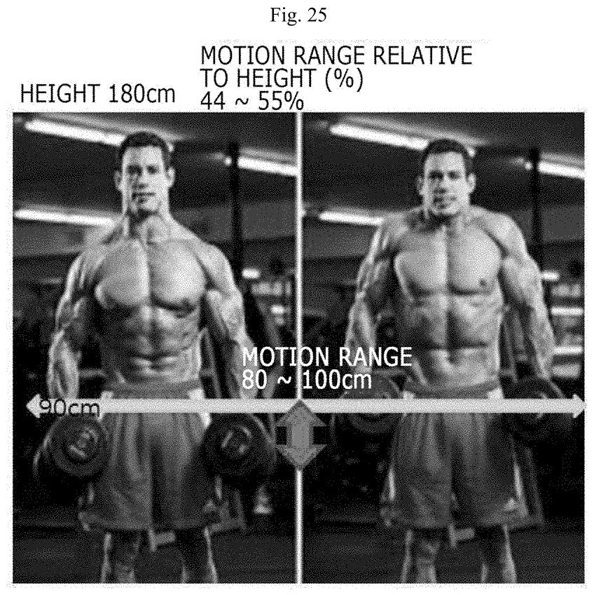

As shown in FIG. 25, the dumbbell shrug is an exercise in which the dumbbell with the ultrasonic sensor 210 attached thereto is moved from the trochanter major to a side of the pelvis. For the dumbbell shrug, the scapulothoracic joint and trapezii are used. In the case where the identification is performed using the depth camera 100, upon free weight training of the dumbbell shrug, only the height of the shoulders is varied in the standing position. The identification may be performed in the range of the moving height by the depth camera 100 or ultrasonic sensor 210. For example, if the user is 180 cm tall, the motion ranges from about 80 cm to about 100 cm, and the ratio of body motion to height ranges from about 44% to about 55%.

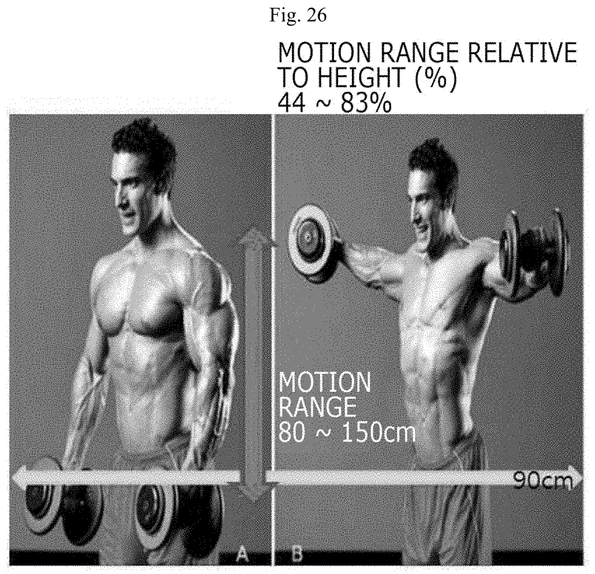

As shown in FIG. 26, the dumbbell lateral raise is an exercise in which the dumbbell with the ultrasonic sensor 210 attached thereto is moved from the trochanter major to the shoulder. For the dumbbell lateral raise, the shoulder joints and deltoid are used. In the case where the identification is performed using the depth camera 100, upon free weight training of the dumbbell lateral raise, both hands are moved up to the shoulders in the standing position, with the elbow joints fixed. The identification may be performed in the range of the moving height by the depth camera 100 or ultrasonic sensor 210. For example, if the user is 180 cm tall, the motion ranges from about 80 cm to about 150 cm, and the ratio of body motion to height ranges from about 44% to about 83%.

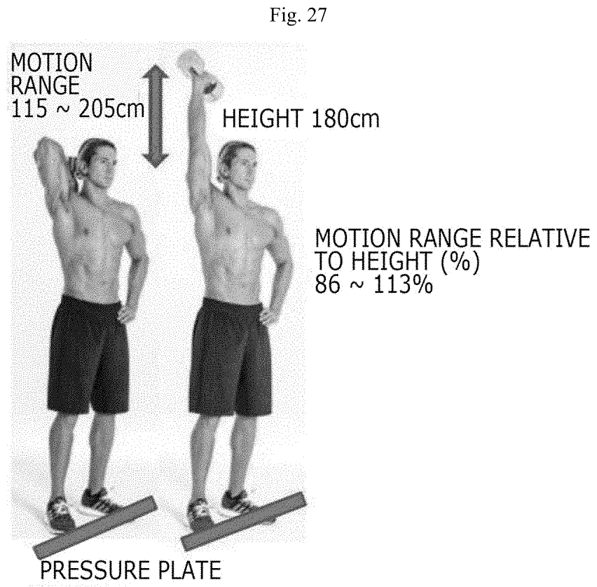

As shown in FIG. 27, the one arm dumbbell triceps extension is an exercise in which the dumbbell with the ultrasonic sensor 210 attached thereto is moved from the center of the back of the head to overhead. For the one arm dumbbell triceps extension, the shoulder and elbow joints and musculus triceps brachii are used. In the case where the identification is performed using the depth camera 100, upon free weight training of the one arm dumbbell triceps extension, one arm is lifted in the standing position and, then, only the height of hand is varied only with an exercise on the elbow joints. The identification may be performed in the range of the moving height by the depth camera 100 or ultrasonic sensor 210. For example, if the user is 180 cm tall, the motion ranges from about 155 cm to about 205 cm, and the ratio of body motion to height ranges from about 86% to about 113%.

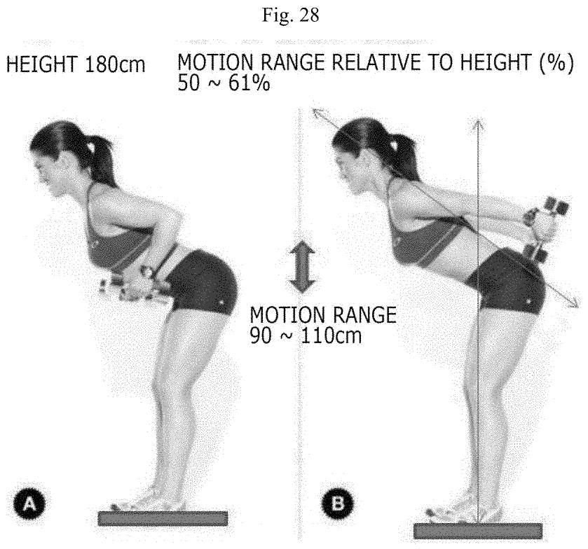

As shown in FIG. 28, the dumbbell kick back is an exercise in which the dumbbell with the ultrasonic sensor 210 attached thereto is moved from side of the pelvis to the lumbar region. For the dumbbell kick back, the elbow joints and musculus triceps brachii are used. In the case where the identification is performed using the depth camera 100, upon free weight training of the dumbbell kick back, the header is lowered and, then, only elbows are extended, varying the height of the hands. The identification may be performed in the range of the moving height by the depth camera 100 or ultrasonic sensor 210. For example, if the user is 180 cm tall, the motion ranges from about 90 cm to about 110 cm, and the ratio of body motion to height ranges from about 50% to about 61%.

The storage unit 400 may store (or manage) the ratio of body motion range to height per exercise type of Table 1 and motion range under the control of the controller 700. In this case, as the height, a preset reference value, e.g., 180 cm, may be used.

TABLE 1

| | motion range | Ratio (%) of motion |
|---|---|---|
| Type of exercise | (cm) | range to height |
| barbell deadlift | 80 to 20 | 44 to 11 |
| bent over barbell row | 80 to 40 | 44 to 22 |
| barbell shoulder press | 150 to 210 | 83 to 116 |
| barbell arm curl | 80 to 150 | 44 to 83 |
| barbell bench press | 70 to 110 | 39 to 61 |
| incline barbell bench press | 90 to 130 | 50 to 72 |
| decline barbell bench press | 60 to 80 | 33 to 44 |
| barbell squat | 150 to 100 | 83 to 55 |
| barbell lunge | 140 to 120 | 78 to 67 |
| barbell shrug | 80 to 100 | 44 to 55 |
| barbell upright row | 80 to 140 | 44 to 77 |
| E-Z bar triceps extension | 150 to 205 | 86 to 113 |
| dumbbell deadlift | 80 to 20 | 44 to 11 |
| bent over dumbbell row | 80 to 40 | 44 to 22 |
| dumbbell shoulder press | 150 to 210 | 83 to 116 |
| alternate dumbbell arm curl | 80 to 150 | 44 to 83 |
| dumbbell bench press | 70 to 110 | 39 to 61 |
| incline dumbbell chest press | 90 to 130 | 50 to 72 |
| decline dumbbell chest press | 60 to 80 | 33 to 44 |
| dumbbell squat | 150 to 100 | 83 to 55 |
| dumbbell lunge | 140 to 120 | 78 to 67 |
| dumbbell shrug | 80 to 100 | 44 to 55 |
| dumbbell lateral raise | 80 to 150 | 44 to 83 |
| one arm dumbbell triceps | 155 to 205 | 86 to 113 |
| extension | | |
| dumbbell kick back | 90 to 110 | 50 to 61 |

The display unit 500 may display various contents, e.g., various menu screens, using the UI and/or GUI stored in the storage unit 400 under the control of the controller 700. The contents displayed on the display unit 500 include a menu screen including various pieces of text or image data (including various information data), icons, a list menu, combo boxes, or other various pieces of data. The display unit 500 may include a touchscreen. The display unit 500 may include at least one of a liquid crystal display (LCD), a thin film transistor-liquid crystal display (TFT-LCD), an organic light-emitting diode (OLED), a flexible display, or a three-dimensional (3D) display, an e-ink display, or a light emitting diode (LED) display. The display unit 500 may display various pieces of information measured by the sensor unit 200 and gathered under the control of the controller 700. The voice output unit 600 outputs voice information included in a predetermined signal signal-processed by the controller 700. The voice output unit 600 may include, e.g., a receiver, a speaker, and a buzzer.

The voice output unit 600 outputs a guidance (or instruction) voice generated by the controller 700.

The voice output unit 600 outputs voice information (or sound effect) corresponding to various pieces of information measured by the sensor unit 200 and gathered by the controller 700.

The controller (or a microcontroller unit (MCU)) 700 may execute an overall control function of the smart free weight training motion recognition device 10.

The controller 700 executes an overall control function of the smart free weight training motion recognition device 10 using the program and data stored in the storage unit 400. The controller 700 may include a RAM, a ROM, a central processing unit (CPU), a graphics processing unit (GPU), and a bus, and the RAM, ROM, CPU, and GPU may be interconnected via the bus. The CPU may access the storage unit 400 and boot the operating system (OS) stored in the storage unit 400. The CPU may perform various operations using various programs, contents, and data stored in the storage unit 400.

The controller 700 outputs, via the display unit 500 and/or the voice output unit 600, user recognition results for the user doing exercise on the piece of exercise equipment recognized by the face recognition sensor 260, the fingerprint recognition sensor 270, and the iris recognition sensor 280.

For example, the controller 700 identifies information about the user doing exercise on the piece of exercise equipment, recognized by the face recognition sensor 260, fingerprint recognition sensor 270, and iris recognition sensor 280 and outputs the identified information (or user recognition result) about the exercising user via the display unit 500 and/or the voice output unit 600.

The controller 700 identifies (or determines/computes/obtains/measures) motion information (or body joint information) about the joints on which the user is doing on the piece of exercise equipment, type of exercise and number of repetitions of exercise, and user's body information, based on variations in the user's body surface (or motion of each joint of the user in the image information) according to the motion of each joint of the user in the obtained (or captured) image information and the height of the section in which the piece of exercise equipment is repeatedly moved (or the range of motion of the section in which the piece of exercise equipment is moved) included in the measured state of the piece of exercise equipment. The exercise type includes a barbell deadlift, a bent over barbell row, a barbell shoulder press, a barbell arm curl, a barbell bench press, an incline/decline barbell bench press, a barbell squat, a barbell lunge, a barbell shrug, a barbell upright row, and E-Z bar triceps extension, which use a barbell, a dumbbell deadlift, dumbbell row, dumbbell shoulder press, alternate dumbbell arm curl, dumbbell bench (or chest) press, Incline/decline dumbbell chest press, dumbbell squat, dumbbell lunges, dumbbell shrugs, dumbbell lateral rises, one arm dumbbell triceps extensions, and dumbbell kickbacks, which use dumbbells. The deadlift, row, shoulder press, arm curl, bench press, incline/decline bench press, squat, lunge, and shrug may be in different states of exercise equipment (e.g., barbell or dumbbell) used for the same exercise motion. Thus, the piece of exercise equipment may be identified via the unique ID recognized from the piece of exercise equipment or image analysis for the images in the image information. The user's body information includes, e.g., the user's height information, position of head, degree of symmetry of the heights of the shoulders and pelvis, knees and elbows, and the length of legs, the length of arms, Q-angle, area of pelvis, area of shoulder, area of abdomen, foot shape, leg shape, per-exercise section foot pressure, position of foot over time during exercise, weight distribution of foot, pressure distribution of foot, and pressure distribution per foot section. The controller 700 may identify (or determine/compute/measure/obtain) the user's body information based on, e.g., variations in the user's body surface (or motion of each joint of the user in the image information) due to the motion of each joint of the user in the image information obtained before the exercise.

For example, the controller 700 measures (or identifies/estimates) the user's height via image processing on the user who is an object in the image information (e.g., pre-exercise and/or mid-exercise image information). The controller 700 may identify the height information in the user-related personal information previously stored in the storage unit 400.

The controller 700 measures the position of the user's head via image processing on the user who is an object in the image information (e.g., the measurement is performed from the front if the median line is used and from a side surface if the gravity line is used). The controller 700 may identify the position of head in the user-related personal information previously stored in the storage unit 400.

The controller 700 measures the degree of symmetry between the heights of the user's shoulders, pelvis, knees, and elbows via image processing on the user who is an object in the image information. The controller 700 may identify the degree of symmetry between the heights of the shoulders, pelvis, knees, and elbows in the user-related personal information previously stored in the storage unit 400.

For example, the controller 700 measures the heights of the user's shoulders, pelvis, knees, and elbows via image processing on the user who is an object in the image information. The controller 700 measures the degree of symmetry between the measured heights of the user's shoulders, pelvis, knees, and elbows.

The controller 700 measures the user's leg length (e.g., femoral region between the pelvis and the knee and calf region between the knee and the ankle) and arm length (e.g., the upper arm between the shoulder and the elbow and the lower arm between the elbow and the wrist) via image processing on the user who is an object in the image information. The controller 700 may identify the leg length and arm length in the user-related personal information previously stored in the storage unit 400.

The controller 700 measures (or determines) the user's Q-angle via image processing on the user who is an object in the image information. The controller 700 may identify the Q-angle in the user-related personal information previously stored in the storage unit 400.

The Q-angle is a relative indicator for recognizing the lateral pull of the quadriceps femoris against the patella. The normal Q-angle may range from 10 degrees to 15 degrees.

The following is a Q-angle measurement method.

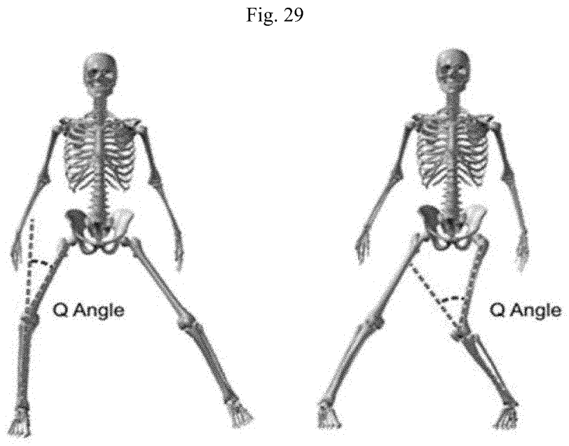

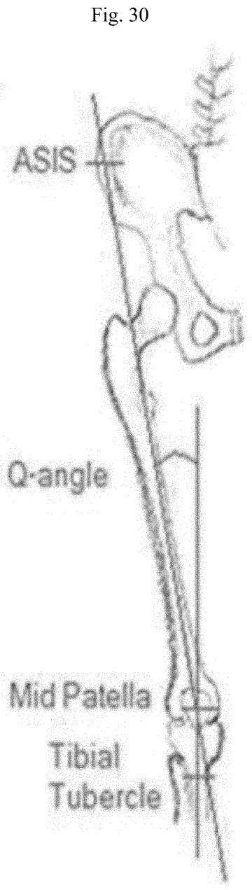

Referring to FIGS. 29 and 30, a straight line is drawn from the user's anterior superior iliac spine (ASIS) to the center line of the patella via image processing on the user who is an object in the image information.

As shown in FIGS. 29 and 30, a straight line is drawn from the center line of the patella and the tibial tubercle.

The angle between the two straight lines is the Q-angle.

The controller 700 measures each of the area of the user's pelvis, area of shoulder, and area of abdomen via image processing on the user who is an object in the image information.

The controller 700 determines the user's obesity level based on the measured pelvis-to-abdomen ratio of the user (or ratio of area of pelvis to area of abdomen). The controller 700 may identify the obesity level in the user-related personal information previously stored in the storage unit 400.

The controller 700 determines the user's body shape based on the measured pelvis-to-shoulder ratio of the user (or ratio of area of pelvis to area of shoulder). The controller 700 may identify the body shape in the user-related personal information previously stored in the storage unit 400.

In determining the obesity level, if a Korean person's waist circumference measures 90 cm (35.4 inches) or more for men and 80 cm (31.5 inches) or more for women, he or she is regarded obese regardless of his/her height and weight, which is called “central obesity.”

Rather than waist circumference alone, the waist-to-hip ratio (WHR) is primarily used which may reflect muscle mass to some degree based on the waist circumference.

Thus, even if one's waist circumstance measures large and thus he is judged as central obesity, if the measurement is small, he may be considered to have a lower health risk.

The waist-to-hip ratio (WHR) is the value resultant from dividing the waist circumstance by the hip circumstance, and the reference to be determined to have abdominal obesity is 0.85 or more for women and 0.9 or more for men.

If the weight is measured using the pressure plate, the obesity level, called the body mass index (BMI) which is the height-to-weight ratio, may be assessed.

As a method for determining obesity, the BMI is the height (kg) divided by the square of the height (m). The BMI well reflects body fat accumulation and is thus widely used to judge obesity.

The BMI is categorized as normal (20 to 25), overweight (obese class I) (25 to 29.9), obese (obese class II) (30 to 40), and severely obese (40.1 or more).

The controller 700 determines the user's foot shape and leg shape via image processing on the user who is an object in the image information and determines whether the legs are normal, bowlegs, or knock-knee based on the identified foot or leg shape. In this case, the controller 700 may identify whether the legs are normal, bowlegs, or know-knee according to the foot and let shapes in the user-related personal information previously stored in the storage unit 400.

The controller 700 generates (or forms) body segment or skeleton images related to the user via image processing on the user who is an object in the image information.

The controller 700 displays the generated skeleton images related to the user before or during exercise on the display unit 500.

As such, the controller 700 may measure the user-related body information in the image information.

The reason for measuring the user's body information is as follows.

For example, a person's femoral region (or thigh) or lower leg region (calf) differs from person to person or there is no absolute ratio. Thus, upon squat exercise, the following cases occur.

Case 1: Where the femoral region is relatively longer than the lower leg region

Case 2: Where the femoral region is shorter than the lower leg region

In both cases or in any case, the stability and muscle strength may be maximized as the body's gravity line is properly maintained during squat exercise. However, to do squat exercise while properly maintaining the gravity line, exercise loads or stress are increased on different lower body portions (including the foot pressure) in cases 1 and 2, due to the difference in length between the femoral region and lower leg region.

This means stress exceeding the normal level and may increase the risk of injury and imbalance in muscular strength between the femoral region and lower leg region.

Thus, if the body's asymmetry or gravity line asymmetry is determined to occur during exercise, based on the user's body information measurement, the controller 700 may identify the problems based on, e.g., the user's body information and joint exercise information, body and/or joint position information, time information per motion section, foot pressure distribution, and per-foot section pressure and may provide feedback information.

The controller 700 measures (or computes/identifies/estimates) the height of section, in which the piece of exercise equipment is repeatedly moved, (or body's motion range/motion range of the section where there is repeated motion) via analysis on the image information.

The controller 700 calculates the user's measured height-to-body motion range ratio.

The controller 700 identifies motion information (or body joint information) about the joints on which the user is doing exercise using the piece of exercise equipment, type of exercise, and number of repetitions of exercise based on the calculated height-to-body motion range ratio, the calculated height of section in which the piece of exercise equipment is repeatedly moved, the user's body and/or joint shape in the image information, and the user's body and/or joint motion type in the image information.

The controller 700 identifies the type of exercise in which the user is doing exercise using the piece of exercise equipment, corresponding to the calculated height-to-body motion range ratio and motion range among per-exercise type height-to-body motion range ratio and motion ranges previously stored in the storage unit 400.

The controller 700 may identify the number of repetitions of the motion type of the user's body and/or joint over time and identify (or compute) the number of repetitions of the piece of exercise equipment on which the user is doing exercise.

The controller 700 identifies motion information (or body joint information) of the joint on which the user is doing exercise using the piece of exercise equipment, exercise type, and number of repetitions of exercise, based on the measured height of the section in which the piece of exercise equipment is repeatedly moved (or the motion range of section in which the piece of exercise equipment is repeatedly moved).

The controller 700 computes the ratio of the motion range as per the measured height (or height range of the section in which the piece of exercise equipment is repeatedly moved to the height in the user-related personal information previously stored in the storage unit 400.

The controller 700 identifies motion information (or body joint information) about the joints on which the user is doing exercise using the piece of exercise equipment, based on the calculated height-to-body motion range ratio, the measured height of section in which the piece of exercise equipment is repeatedly moved, and the measured shape of repeated motion of the piece of exercise equipment.

The controller 700 identifies the type of exercise in which the user is doing exercise using the piece of exercise equipment, corresponding to the calculated height-to-body motion range ratio and motion range and the measured height range of the section in which the piece of exercise equipment is repeatedly moved among per-exercise type height-to-body motion range ratio and motion ranges previously stored in the storage unit 400.

The controller 700 may identify the number of repetitions of the motion type of the user's body and/or joint over time and identify (or compute) the number of repetitions of the piece of exercise equipment on which the user is doing exercise.

If only a specific joint is determined to be moved based on the image information obtained via the depth camera 100, the controller 700 may identify the type of exercise which is the user is doing on the piece of exercise equipment.

For example, the controller 700 may identify that the user is doing a shoulder press, based on the image information obtained via the depth camera 100, which shows that the user stands, moves his shoulders and elbows, and lifts his hands overhead.

As another example, the controller 700 may identify that the user is doing a bent over row, based on the image information obtained via the depth camera 100, which shows that the user moves his shoulders and elbows, with the head lowered and hands positioned in front of the pelvis.

As such, the controller 700 may identify the motion information (or body joint information) about the joints on which the user is doing exercise using the piece of exercise equipment, type of exercise and number of repetitions of exercise, and user's body information, based on the obtained image information and/or the measured exercise equipment state information.

The controller 700 identifies the kind and weight of the piece of exercise equipment on which the user is doing exercise, corresponding to unique identifiers, based on the unique identifier attached to the piece of exercise equipment, in the obtained (or captured) image information, the unique identifier attached to the piece of exercise equipment recognized via the RFID reader 220, the magnitude of magnetic field measured via the magnetic sensor 240, and the shape and length of the piece of exercise equipment in the obtained image information.

For example, the controller 700 recognizes the unique identifier attached to the piece of exercise equipment in the image information via image processing on the image information. The controller 700 identifies the kind and weight information of the piece of exercise equipment corresponding to the recognized unique identifier, of per-unique identifier exercise equipment kind and weight information previously stored in the storage unit 400. The controller 700 may identify the characteristics (or feature points) of the piece of exercise equipment which is an object in the image information via image processing on the image information and identify the kind of piece of exercise equipment based on the identified characteristics (or feature points) of the piece of exercise equipment. The kind of piece of exercise equipment includes barbells, dumbbells (or kettlebells), or E-Z bars.

The controller 700 identifies the kind and weight of piece of exercise equipment corresponding to the unique identifier attached to the piece of exercise equipment, identified via the RFID reader 220.

For example, the controller 700 identifies the kind and weight information of the piece of exercise equipment corresponding to the unique identifier attached to the piece of exercise equipment, recognized via the RFID reader 220, of per-unique identifier exercise equipment kind and weight information previously stored in the storage unit 400.

The controller 700 identifies the weight of the piece of exercise equipment corresponding to the magnitude of magnetic field measured via, e.g., the hall sensor 230 or magnetic sensor 240.

For example, the controller 700 identifies the weight of the piece of exercise equipment corresponding to the magnitude of magnetic field measured by the hall sensor 230 or magnetic sensor 240, of per-exercise equipment weight magnetic field magnitude information previously stored in the storage unit 400.

The controller 700 identifies the shape and length of the piece of exercise equipment in the image information via image processing on the image information. The controller 700 identifies the kind and weight information of the piece of exercise equipment corresponding to the identified shape and length of piece of exercise equipment, of weight information according to the shapes and lengths of exercise equipment previously stored in the storage unit 400. The controller 700 may identify the characteristics (or feature points) of the piece of exercise equipment which is an object in the image information via image processing on the image information and identify the kind of piece of exercise equipment based on the identified characteristics (or feature points) of the piece of exercise equipment.

As such, the controller 700 may identify the kind and weight of the piece of exercise equipment which the user is using during exercise based on the obtained image information and/or the measured exercise equipment state information.

The controller 700 computes (or measures/identifies) variations in the user's body median line and gravity line (or center line) per exercise section over time, of the user doing exercise using the piece of exercise equipment, based on the obtained (or captured) image information. In this case, the controller 700 may use the image information captured or obtained in front of the user and image information captured or obtained on the side surface of the user to compute the variations in the body median line and gravity line (or center line). The body gravity line (or center line) may be measured on the side surface of the body, and the median line of the body may be measured in front of the body.

Ideal exercise techniques are able to lift the maximum weight, and abnormal activities of the body median line and gravity line (or center line) are unable to lift the maximum weight, are the cause of acute injury (e.g., upon imbalance), and cause an increase in repeated stress in a specific region and hence chronic injuries.

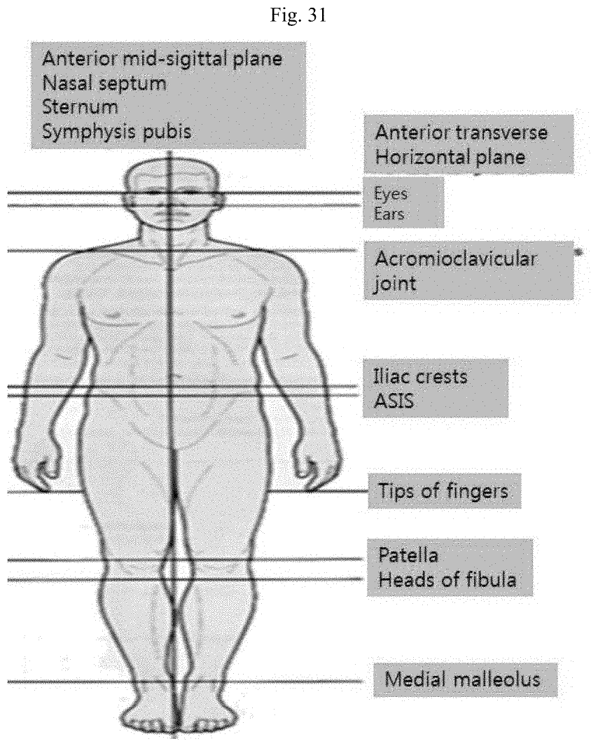

As shown in FIG. 31, the body median line divides the body into the left and right.

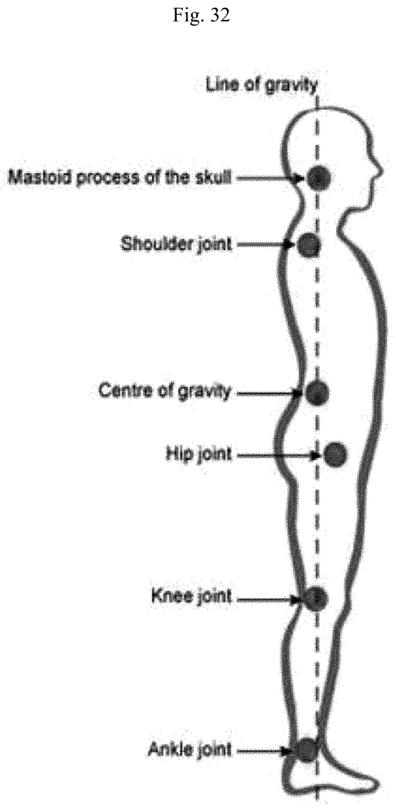

As shown in FIG. 32, the body center line and gravity line, which is the center of gravity or the center of body mass, is where all parts are weight-balanced, and its position relies on many factors, such as the spatial size of body parts, fat and muscle distributions, posture, structural malformation, or external forces.

In the average human body, the center of gravity is the point above the median line just before the level of the second sacrum.

The line of gravity is a vertical line passing through the center of gravity. The line of gravity acting on a person standing in an ideal position passes the mastoid process, the front of the second sacral vertebra, just behind the hip joint, and before the knee joint, ankle joint, or the talocrural joint.

Further, the gravity passing behind the waist creates a constant extension torque to the waist, promoting a natural lordosis curve. In contrast, the gravity passing in front of the waist creates a constant flexion torque. In both cases, the external torque generated by the gravity line should be neutralized by forces and torques actively produced by the muscles and passively produced by the connective tissues.

Abnormal curvatures change the relationship between the gravity line and each vertebral area, causing a decrease in exercise performance and injury.

In the case of abnormal spinal curvature, damage may be caused by increased stress on muscles, ligaments, bones, vertebral discs, facet joints, and spinal nerve roots.

Based on the gravity line and the median line dividing the human body to the left and right, upon exercise, the joint asymmetry between front and back/left and right is measured, and measurement of the foot pressure and comparison of per-section foot pressures may be performed via the foot pressure upon exercise.

Further, where the center of mass coincides with the line of gravity upon exercise, the exercise may be performed stably and at a high weight.

In other words, the controller 700 may feed back information, such as a proper posture, to the user currently doing exercise using the piece of exercise equipment, based on the fact that the user may lift more weight and be less injured in the context where the line of gravity is as close to the weight as possible, upon exercise using the gravity line (or center line) and median line related to the user doing exercise using the identified piece of exercise equipment.

Further, the position of the center of gravity of the barbell may be inferred by the position of foot press being at the front or rear.

The controller 700 identifies the position of foot over time upon exercise, weight distribution of feet (e.g., the left foot and right foot), pressure distribution of foot, and per-foot section pressures (e.g., pressure distribution in the front/back/left/right sections or per-foot section pressure distribution).

Further, the controller 700 identifies (or computes/measures) the per-exercise section exercise types of each joint over time upon exercise, total plantar pressure, and per-exercise section plantar pressures, based on the per-exercise section plantar pressures for the motion of each joint and/or body over time, measured via the pressure plate 250.

That is, the controller 700 divides the foot pressure into a plurality of sections, such as front, back, left, and right and analyzes the distribution of pressure applied to each of both feet in each of the plurality of sections. The controller 700 may analyze the distribution of pressure applied to each of both feet per exercise section over time.

The controller 700 computes (or measures) dynamical elements of each joint based on the motion of each joint (or per-section exercise types of each joint/variations in the user's body surface according to the motion of each joint) over time upon exercise according to values measured via the sensor unit 200. In this case, the motion of each joint includes information, such as the phase, angle, speed, displacement, or depth of each joint over time, measured by the sensor unit 200. The dynamical elements of each joint include momentum, impact, torque, moment of inertia, centripetal force, work, power, kinetic energy, potential energy, angular momentum, and centripetal acceleration.

The controller 700 computes the dynamical elements of each joint, including the momentum, which is the product of mass and velocity, the amount of impact, which is the product of force and action time, the torque, which is the product of force and moment arm, the moment of inertia, which is the product of mass and the square of the radius of rotation, the centripetal force, which is the product of mass and centripetal acceleration, the work (w), which is the product of force and horizontal travel distance, power, which is the product of force and speed, kinetic energy, which is based on mass and velocity, potential energy, which is based on mass, height, and gravitational acceleration, angular momentum, which is the product of momentum of inertia and angular velocity, and centripetal acceleration, which is the product of rotation radius and angular velocity, based on the measured motion of each joint over time (including, e.g., the phase, angle, speed, displacement, and depth of the joint)

The controller 700 identifies (or recognizes) the free weight training motion which the user is doing using the piece of exercise equipment, based on, e.g., the identified joint motion information (or body joint information), exercise type and number of repetitions of exercise, the identified kind and weight of piece of exercise equipment, the identified foot position, the weight distribution of foot, pressure distribution of foot, and per-foot section pressures (or per-foot section pressure distribution).

The controller 700 compares standard posture information as per exercise types preset per piece of exercise equipment, pre-stored in the storage unit 400, with the identified user body information, joint motion information (or body joint information), exercise type and weight of piece of exercise equipment as per the number of repetitions of exercise, foot position, weight distribution of foot, foot pressure distribution, and per-foot section pressure distribution. The standard posture information includes body information corresponding to the optimal exercise course and an athlete with superior performance (e.g., a gold medalist in an international game), joint motion information related to the free weight training motion corresponding to the athlete, the number of repetitions as per the weight and exercise type of piece of exercise equipment, foot position, foot weight distribution, foot pressure distribution, and per-foot section pressure distribution.

The controller 700 compares standard posture information as per exercise types preset per piece of exercise equipment, pre-stored in the storage unit 400, with the identified user body information, joint exercise information, body and/or joint position information, per-section time information of motion upon exercise, foot pressure distribution, and per-foot section pressure distribution.

Strength exercise is a comprehensive term that includes one's strength, muscle power, and endurance training. The strength exercise is expressed as the maximum muscle strength (1 RM), meaning repeated exercise of muscle strength (for example, 6 or less), muscle hypertrophy (for example, 6 to 12), and muscle endurance (for example, 12 or more).

Where the user sets the target exercise (or target amount/training target) as muscle hypertrophy, if the identified number of repetitions does not match the target number, the controller 700 outputs notification information via the display unit 500 and/or the voice output unit 600.

As such, measurement of a proper posture of exercise and the number of repetitions of exercise may aid in maximizing the efficiency of exercise and achieving the exercise target in a safer and quicker manner.

The exercise loads and repeat count according to the training target may be set as shown in Table 2.

TABLE 2

| | | target | | |
|---|---|---|---|---|
| | load | repeat | | |
| training target | (% 1 RM) | count | set | break time |
| muscle strength | >=85 | <=6 | 2 to 6 | 25 minutes |
| explosive power | | | | |
| single occasion | 80 to 90 | 1 to 2 | 3 to 5 | 2 to 5 minutes |
| repeated occasion | 75 to 85 | 3 to 5 | 3 to 5 | 2 to 5 minutes |
| muscle | 67 to 85 | 6 to 12 | 3 to 6 | 30 seconds to |
| hypertrophy | | | | 90 seconds |
| muscle | >=67 | >=12 | 2 to 3 | 30 seconds or |
| endurance | | | | less |

For example, if one is doing muscle hypertrophy, his maximum muscle strength (1 RM) means the weight he can lift up to once. In the case where it may be repeated once using 100 kg, to set a repeat count of six times to 12 times for muscle hypertrophy, this represents that it may be repeated 12 times using a weight of 67% of the maximum muscle strength and it may be repeated six times using 85%. The controller 700 generates feedback information related to the user's free weight training motion according to the result of comparison. The feedback information includes, e.g., the optimal exercise course information which is information customized to the user, information about whether the user's exercise goal is achieved or not, the dynamical element of each joint (or dynamical element of body) according to the user's exercise goal, information for partial reinforcing exercise, information for retraining exercise technique (including, e.g., information about the body and/or joint motion or posture, foot position information, foot weight distribution information, foot pressure distribution information, and per-foot section pressure distribution), comparison image information for standard posture information and the foot position and the motion of each joint and/or body of the captured user, user recognition result (or user information), and user's body information. The optimal exercise course information includes, e.g., body posture information according to a performing of the exercise, using the obtained gravity line (or center line) and body median line of the user per exercise section over time. The information about whether the user's exercise goal is achieved includes information about whether the user has normally met the target according to the exercise goal according to the result of comparison and the exercise performing rate according to the exercise goal. The exercise goal may target reinforcing muscle strength, explosive power, muscle hypertrophy, and muscle endurance.

The controller 700 outputs the generated feedback information via the display unit 500 and/or the voice output unit 600.

The controller 700 stores the measured user-related body information, joint motion information (or body joint information), weight of piece of exercise equipment according to the exercise type and exercise repeat count, foot position, foot weight distribution, foot pressure distribution, per-foot section pressure distribution, and feedback information in a cloud server (not shown), where the user has enrolled or subscribed, via the communication unit 300.

The controller 700 may transmit the measured user-related body information, joint motion information (or body joint information), weight of piece of exercise equipment according to the exercise type and exercise repeat count, foot position, foot weight distribution, foot pressure distribution, per-foot section pressure distribution, and feedback information, to a terminal (not shown) related to the user via the communication unit 300 or may store (or register) the information in a social network service.

As such, various pieces of information, such as motion for, e.g., the phase, angle, speed, displacement, and depth of major body joints of the user doing exercise using the piece of exercise equipment, exercise type, exercise repeat count, and exercise weight may be recognized using the depth camera or various sensors, and the free weight training motion which the user is doing on the piece of exercise equipment may be recognized based on the recognized pieces of information.

Feedback for the user's body information, reeducation of exercise technique, and partial reinforcing exercise may be provided via comparison between pre-stored pieces of information related to the free weight training motion corresponding to an athlete with superior performance and the optimal exercise course with pieces of information gathered in relation to the free weight training motion the user is doing using the piece of exercise equipment.

A smart free weight training motion recognition method according to an embodiment of the disclosure is described below in detail with reference to FIGS. 1 to 34.

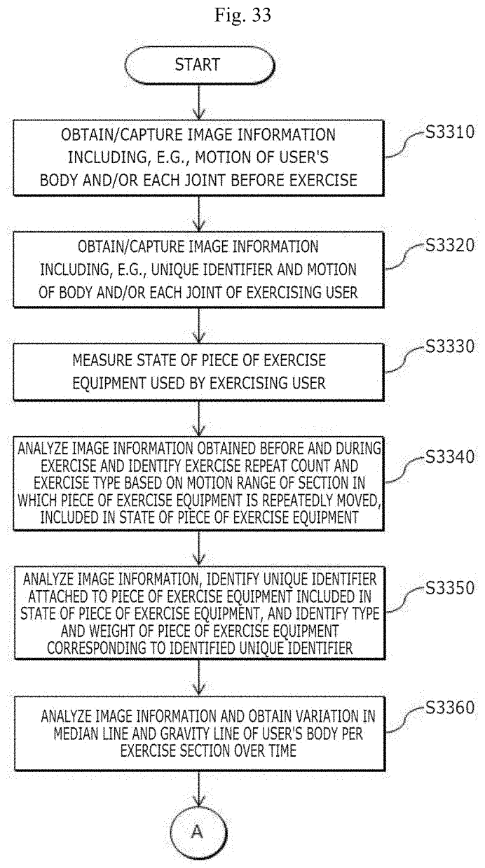

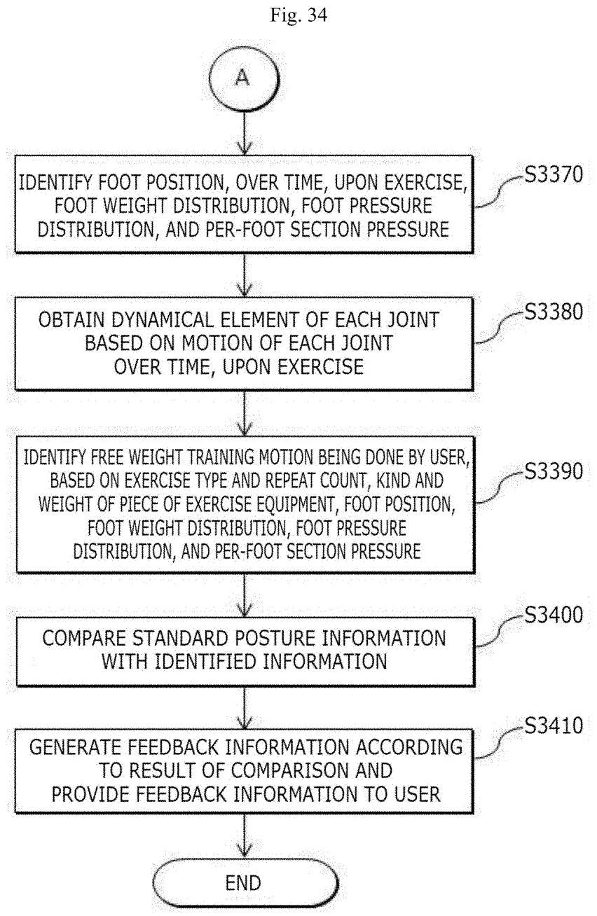

FIGS. 33 and 34 are flowcharts illustrating a smart free weight training motion recognition method according to an embodiment of the disclosure.

The depth camera 100 may obtain or capture image information (or pre-exercise image information) including, e.g., the motion of the user's body and/or each joint before exercise (or variations in the user's body surface due to motion of the user's body and/or each joint before exercise). The variations in the user's body surface due to the motion of each joint include, e.g., the phase, angle, speed, displacement, and depth due to the joint motion. The image information may be image information resultant from capturing the motion of the user's body and/or joint on the front surface and/or side surface of the user doing exercise on the piece of equipment. The depth camera 100 may obtain voice information around the user, as well as the image information.

As an example, before the user starts exercise, the depth camera 100 obtains third image information including, e.g., the variations in the user's body surface due to the motion of the user's body and/or each joint in front of the user and obtains fourth image information including, e.g., variations in the user's body surface due to the motion of the user's body and/or each joint from a side surface (S3310).

Thereafter, the depth camera 100 obtains (or captures) image information including the motion of each joint and/or the body of the user doing exercise on the piece of exercise equipment (or variations in the user's body surface due to the motion of each joint and/or the body of the exercising user) and the unique identifier (or unique image ID) attached to the piece of exercise equipment. The variations in the user's body surface due to the motion of each joint include, e.g., the phase, angle, speed, displacement, and depth due to the joint motion. The piece of exercise equipment includes barbells, dumbbells (or kettlebells), or E-Z bars. The unique ID may include a difference piece of information depending on the kind or weight of the piece of equipment. The image information may be image information resultant from capturing the motion of the user's body and/or joint on the front surface and/or side surface of the user doing exercise on the piece of equipment. The depth camera 100 may obtain voice information around the user, as well as the image information.

As an example, the depth camera 100 obtains first image information including, e.g., the variations in the user's body surface due to the motion of the user's body and/or each joint in front of the user doing exercise using a first piece of exercise equipment and a first unique identifier attached to the first piece of exercise equipment and obtains second image information including, e.g., variations in the user's body surface due to the motion of the user's body and/or each joint from a side surface and the first unique identifier attached to the first piece of exercise equipment (S3320).

The sensor unit 200 measures the state of the piece of exercise equipment on which the user is doing exercise and recognizes the user doing exercise on the piece of exercise equipment. The state of the piece of exercise equipment (or the state information about the piece of exercise equipment) may include information about the variation in height due to the time variation in the piece of exercise equipment over time as the piece of exercise equipment is used by the exercising user, the unique identifier of the piece of exercise equipment, magnitude of magnetic field, and per-exercise section foot pressure.

For example, the ultrasonic sensor 210 included in the sensor unit 200 measures the height of the section in which the piece of exercise equipment is repeatedly moved by the user doing exercise using the piece of exercise equipment (or the range of motion of the section of motion).

The RFID reader 220 included in the sensor unit 200 recognizes the unique ID attached to the piece of exercise equipment on which the user is doing exercise. The piece of exercise equipment may be in the state of different unique IDs (or tags including different unique IDs) attached depending on weights and types. The tags including different unique IDs may be attached to, or detached from, the piece of exercise equipment. Where the piece of exercise equipment on which the user is doing exercise is a barbell, dumbbell, or E-Z bar which has (fixed) weights attached at the left and right, the RFID reader 220 may recognize each of the unique IDs in the tags individually attached to the left and right weights or recognizes the unique ID in one tag attached to one of the left and right weights. If the RFID reader 220 individually recognizes the unique IDs from the tags attached to the left and right weights, the weight of the piece of exercise equipment may be calculated by summating the weight information included in the recognized unique IDs (e.g., weight information about each of the left and right weights). If the RFID reader 220 recognizes the unique ID from one tag attached to one of the left and right weights, the weight of the piece of exercise equipment may be obtained based on the weight information (e.g., the weight information obtained by summating the left and right weights) included in the unique ID recognized from the tag.

The hall sensor 230 or magnetic sensor 240 included in the sensor unit 200 measures the magnitude of the magnetic field for the magnet attached to the piece of exercise equipment on which the user is doing exercise. A magnets with a different magnitude of magnetic field depending on the weight may be attached to one side of the piece of exercise equipment. The distance between the hall sensor 230 or magnetic sensor 240 and the magnet attached to the piece of exercise equipment may be positioned at a radius within a preset error range, so that accuracy may be increased. The pressure plate 250 included in the sensor unit 200 measures the per-exercise section foot pressure for the motion of the body and/or each joint, over time, for the user doing exercise on the piece of exercise equipment. The exercise section refers to the state in which for a series of exercises the user is doing, specific muscles or joint angles used per step differ.

The face recognition sensor 260, fingerprint recognition sensor 270, or iris recognition sensor 280 configured or provided on a side of the piece of exercise equipment and included in the sensor unit 200 recognizes the user doing exercise using the piece of exercise equipment

As an example, as shown in FIG. 6, the ultrasonic sensor 210 measures the height (e.g., a range from 150 cm to 210 cm from the ground) of the section in which the barbell is repeated moved by the user doing exercise using the barbell.

The RFID reader 220 recognizes a first unique identifier (e.g., including information such as barbell and 5 kg) in one tag attached to one of the left and right sides of the barbell.

The magnetic sensor 240 measures a first magnetic field magnitude for a first magnet attached to the barbell.

As shown in FIG. 6, the pressure plate 250 measures the per-exercise section foot pressure for the motion of the body and/or each joint, over time, for the user doing exercise using the barbell.

The fingerprint recognition sensor 270 attached to one side of the barbell recognizes the fingerprint of the user doing exercise using the barbell and recognizes (or identifies) the user (S3330).

The controller 700 identifies (or determines/computes/obtains/measures) motion information (or body joint information) about the joints on which the user is doing on the piece of exercise equipment, type of exercise and number of repetitions of exercise, and user's body information, based on variations in the user's body surface (or motion of each joint of the user in the image information) according to the motion of each joint of the user in the obtained (or captured) image information and the height of the section in which the piece of exercise equipment is repeatedly moved (or the range of motion of the section in which the piece of exercise equipment is moved) included in the measured state of the piece of exercise equipment. The exercise type includes a barbell deadlift, a bent over barbell row, a barbell shoulder press, a barbell arm curl, a barbell bench press, an incline/decline barbell bench press, a barbell squat, a barbell lunge, a barbell shrug, a barbell upright row, and E-Z bar triceps extension, which use a barbell, a dumbbell deadlift, dumbbell row, dumbbell shoulder press, alternate dumbbell arm curl, dumbbell bench (or chest) press, Incline/decline dumbbell chest press, dumbbell squat, dumbbell lunges, dumbbell shrugs, dumbbell lateral rises, one arm dumbbell triceps extensions, and dumbbell kickbacks, which use dumbbells. The deadlift, row, shoulder press, arm curl, bench press, incline/decline bench press, squat, lunge, and shrug may be in different states of exercise equipment (e.g., barbell or dumbbell) used for the same exercise motion. Thus, the piece of exercise equipment may be identified via the unique ID recognized from the piece of exercise equipment or image analysis for the images in the image information. The user's body information includes, e.g., the user's height information, position of head, degree of symmetry of the heights of the shoulders and pelvis, knees and elbows, and the length of legs, the length of arms, Q-angle, area of pelvis, area of shoulder, area of abdomen, foot shape, leg shape, per-exercise section foot pressure, position of foot over time during exercise, weight distribution of foot, pressure distribution of foot, and pressure distribution per foot section. The controller 700 may identify (or determine/compute/measure/obtain) the user's body information based on, e.g., variations in the user's body surface (or motion of each joint of the user in the image information) due to the motion of each joint of the user in the image information obtained before the exercise.

For example, the controller 700 measures (or identifies/estimates) the user's height via image processing on the user who is an object in the image information (e.g., pre-exercise and/or mid-exercise image information). The controller 700 may identify the height information in the user-related personal information previously stored in the storage unit 400. The controller 700 measures (or identifies/estimates), e.g., the position of the user's head, the degree of symmetry of the heights of shoulders, pelvis, knees, and elbows, leg length, arm length, Q-angle, area of pelvis, area of shoulder, area of abdomen, foot shape, and leg shape via image processing on the user who is an object in the image information. In this case, the controller 700 may also identify the degree of symmetry of the heights of the shoulders, pelvis, knees, and elbows, leg length, arm length, Q-angle, area of pelvis, area of shoulder, area of abdomen, foot shape, and leg shape in personal information related to the user previously stored in the storage unit 400 (or personal information related to the recognized user among a plurality of pieces of personal information pre-stored in the storage unit 400). The controller 700 determines the user's obesity level based on the pelvis-to-abdomen ratio (or ratio of area of pelvis to area of abdomen). The controller 700 determines the user's body shape based on the pelvis-to-shoulder ratio of the user (or ratio of area of pelvis to area of shoulder). The controller 700 identifies the foot shape and leg shape, determining whether the legs are normal, bowlegs, or knock-knee. The controller 700 measures (or computes/identifies/estimates) the height of section, in which the piece of exercise equipment is repeatedly moved, (or body's motion range/motion range of the section where there is repeated motion) via analysis on the image information. The controller 700 calculates the user's measured height-to-body motion range ratio. The controller 700 identifies motion information (or body joint information) about the joints on which the user is doing exercise using the piece of exercise equipment, type of exercise, and number of repetitions of exercise based on the calculated height-to-body motion range ratio, the calculated height of section in which the piece of exercise equipment is repeatedly moved, the user's body and/or joint shape in the image information, and the user's body and/or joint motion type in the image information. For example, the controller 700 identifies the motion of the user's body joint (or joint motion information) via image processing on the user who is an object in the image information. The controller 700 identifies the type of exercise in which the user is doing exercise using the piece of exercise equipment, corresponding to the calculated height-to-body motion range ratio and motion range among per-exercise type height-to-body motion range ratio and motion ranges previously stored in the storage unit 400. The controller 700 may identify the number of repetitions of the motion type of the user's body and/or joint over time and identify (or compute) the number of repetitions of the piece of exercise equipment on which the user is doing exercise.

The controller 700 identifies the type and repeat count of the exercise which the user is doing using the piece of exercise equipment, based on the measured height of the section in which the piece of exercise equipment is repeatedly moved (or the motion range of section in which the piece of exercise equipment is repeatedly moved).

The controller 700 computes the ratio of the motion range as per the measured height (or height range of the section in which the piece of exercise equipment is repeatedly moved to the height in the user-related personal information previously stored in the storage unit 400. The controller 700 identifies the type and repeat count of the exercise which the user is doing using the piece of exercise equipment, based on the calculated height-to-body motion range ratio, the measured height of section in which the piece of exercise equipment is repeatedly moved, and the measured shape of repeated motion of the piece of exercise equipment. The controller 700 identifies the type of exercise in which the user is doing exercise using the piece of exercise equipment, corresponding to the calculated height-to-body motion range ratio and motion range and the measured height range of the section in which the piece of exercise equipment is repeatedly moved among per-exercise type height-to-body motion range ratio and motion ranges previously stored in the storage unit 400. The controller 700 may identify the number of repetitions of the motion type of the user's body and/or joint over time and identify (or compute) the number of repetitions of the piece of exercise equipment on which the user is doing exercise.

As such, the controller 700 may identify the motion information (or body joint information) about the joints on which the user is doing exercise using the piece of exercise equipment, type of exercise and number of repetitions of exercise, and user's body information, based on the obtained image information and/or the measured exercise equipment state information.

As an example, the controller 700 measures, e.g., the user's height (e.g., 180 cm), the position of the user's head from the front or side surface, the degree of symmetry of the heights of the user's shoulders, pelvis, knees, and elbows (or symmetry information between the heights of the user's shoulders and pelvis and the heights of knees and elbows), leg length, arm length, Q-angle, obesity level according to the ratio of the area of pelvis to the area of abdomen, body shape according to the ratio of the area of pelvis to the area of shoulder, and kind of leg according to the foot and leg shapes via image processing on the user in the obtained first image information to fourth image information.

The controller 700 identifies the user's motion range (or the range of motion of the section in which the first piece of exercise equipment is repeatedly moved) (e.g., 150 cm to 210 cm from the ground) in the first image information via image processing on the obtained first image information (or the second image information) and computes or obtains the ratio of body motion range to the user's height measured (e.g., 83% to 116% corresponding to 150 cm to 210 cm and 180 cm).

The controller 700 identifies the shoulder press of the free weight training, which the user is doing using the first piece of exercise equipment, based on the obtained height-to-body motion range ratio (e.g., 83% to 116%), the user's motion range (e.g., 150 cm to 210 cm), and the user's body and/or joint motion type in the first image information and/or second image information and identifies the exercise repeat count (e.g., 10 times) via analysis of the first image information (or second image information).

As another example, the controller 700 identifies the height (e.g., 180 cm), position of the user's head, degree of symmetry of the heights of the user's shoulders and pelvis, knees, and elbows, leg length, arm length, Q-angle, obesity level according to the ratio of the area of pelvis to the area of abdomen, the body shape according to the ratio of the area of pelvis to the area of shoulder, and type of legs according to the shape of arms and legs, in the user-related personal information previously stored in the storage unit 400 and computes or obtains the height-to-body motion range ratio (e.g., 83% to 116% corresponding to 150 cm to 210 cm and 180 cm) which is the ratio of the user's height identified to the height (e.g., 150 cm to 210 cm) of the section in which the first piece of exercise equipment is repeatedly moved by the user doing exercise using the first piece of exercise equipment, measured via the ultrasonic sensor 210.

The controller 700 identifies the shoulder press of free weight training which the user is doing using the first piece of exercise equipment, based on the calculated height-to-body motion range ratio (e.g., 83% to 116%), the height (e.g., 150 cm to 210 cm) of the section, in which the first piece of exercise equipment is repeatedly moved, measured via the ultrasonic sensor 210, and the type of the repeated motion of the first piece of exercise equipment measured via the ultrasonic sensor 210 and identifies the exercise repeat count (e.g., 10 times) via analysis of variations in the height of section in which the first piece of exercise equipment is repeatedly moved, measured via the ultrasonic sensor 210 (S3340).

Thereafter, the controller 700 identifies the kind and weight of the piece of exercise equipment on which the user is doing exercise, corresponding to unique identifiers, based on the unique identifier attached to the piece of exercise equipment, in the obtained (or captured) image information, the unique identifier attached to the piece of exercise equipment recognized via the RFID reader 220, the magnitude of magnetic field measured via the magnetic sensor 240, and the shape and length of the piece of exercise equipment in the obtained image information.

For example, the controller 700 recognizes the unique identifier attached to the piece of exercise equipment in the image information via image processing on the image information. The controller 700 identifies the kind and weight information of the piece of exercise equipment corresponding to the recognized unique identifier, of per-unique identifier exercise equipment kind and weight information previously stored in the storage unit 400. The controller 700 may identify the characteristics (or feature points) of the piece of exercise equipment which is an object in the image information via image processing on the image information and identify the kind and weight of piece of exercise equipment based on the identified characteristics (or feature points) of the piece of exercise equipment. The kind of piece of exercise equipment includes barbells, dumbbells (or kettlebells), or E-Z bars.

The controller 700 identifies the kind and weight of piece of exercise equipment corresponding to the unique identifier attached to the piece of exercise equipment, identified via the RFID reader 220.

For example, the controller 700 identifies the kind and weight information of the piece of exercise equipment corresponding to the unique identifier attached to the piece of exercise equipment, recognized via the RFID reader 220, of per-unique identifier exercise equipment kind and weight information previously stored in the storage unit 400.

The controller 700 identifies the weight of the piece of exercise equipment corresponding to the magnitude of magnetic field measured via, e.g., the hall sensor 230 or magnetic sensor 240.

For example, the controller 700 identifies the weight of the piece of exercise equipment corresponding to the magnitude of magnetic field measured by the hall sensor 230 or magnetic sensor 240, of per-exercise equipment weight magnetic field magnitude information previously stored in the storage unit 400.

The controller 700 identifies the shape and length of the piece of exercise equipment in the image information via image processing on the image information. The controller 700 identifies the kind and weight information of the piece of exercise equipment corresponding to the identified shape and length of piece of exercise equipment, of weight information according to the shapes and lengths of exercise equipment previously stored in the storage unit 400. The controller 700 may identify the characteristics (or feature points) of the piece of exercise equipment which is an object in the image information via image processing on the image information and identify the kind and weight of piece of exercise equipment based on the identified characteristics (or feature points) of the piece of exercise equipment.

As such, the controller 700 may identify the kind and weight of the piece of exercise equipment which the user is using during exercise based on the obtained image information and/or the measured exercise equipment state information.

As an example, the controller 700 recognizes a first unique identifier attached to a first piece of exercise equipment in the obtained first image information and identifies the kind (e.g., barbell) and weight (e.g., 5 kg) information of the piece of exercise equipment corresponding to the recognized first unique identifier, of per-unique identifier exercise equipment kind and weight information previously stored in the storage unit 400.

As another example, the controller 700 identifies the kind (e.g., barbell) and weight (e.g., 5 kg) information of the piece of exercise equipment corresponding to the first unique identifier attached to the first piece of exercise equipment, recognized via the RFID reader 220, of per-unique identifier exercise equipment kind and weight information previously stored in the storage unit 400.

As another example, the controller 700 identifies the shape and length of the first piece of exercise equipment in the obtained first image information, identifies the kind (e.g., barbell) of the piece of exercise equipment corresponding to the identified shape of the first piece of exercise equipment among kinds of exercise equipment pre-stored in the storage unit 400, and identifies weight (e.g., 5 kg) information corresponding to the identified shape and length of the first piece of exercise equipment, of the weight information according to the shapes and lengths of exercise equipment pre-stored in the storage unit 400 (S3350).

Thereafter, the controller 700 computes (or measures/identifies) variations in the user's body median line and gravity line (or center line) per exercise section over time, of the user doing exercise using the piece of exercise equipment, based on the obtained (or captured) image information. In this case, the controller 700 may use the image information captured or obtained in front of the user and image information captured or obtained on the side surface of the user to compute the variations in the body median line and gravity line (or center line).

As an example, the controller 700 computes or obtains variations in body median line and gravity line (or center line) per exercise section over time, of the user doing exercise using the first piece of exercise equipment based on the obtained first image information and second image information (S3360).

Thereafter, the controller 700 identifies the position of foot over time upon exercise, weight distribution of feet (e.g., the left foot and right foot), pressure distribution of foot, and per-foot section pressures (e.g., pressure distribution in the front/back/left/right sections or per-foot section pressure distribution).

Further, the controller 700 identifies (or computes/measures) the per-exercise section exercise types of each joint over time upon exercise, total plantar pressure, and per-exercise section plantar pressures, based on the per-exercise section plantar pressures for the motion of each joint and/or body over time, measured via the pressure plate 250.

As an example, the controller 700 identifies each of the position of foot per exercise section over time upon exercise, foot weight distribution, foot pressure distribution, per-foot section pressure distribution, per-exercise section exercise type of each joint, and total plantar pressure, and per-exercise section plantar pressure based on the foot pressure applied to the feet of the user doing exercise using the first piece of exercise equipment, e.g., barbell (S3370).

Thereafter, the controller 700 computes (or measures) dynamical elements of each joint based on the motion of each joint (or per-section exercise types of each joint/variations in the user's body surface according to the motion of each joint) over time upon exercise according to values measured via the sensor unit 200. In this case, the motion of each joint includes information, such as the phase, angle, speed, displacement, or depth of each joint over time, measured by the sensor unit 200. The dynamical elements of each joint include momentum, impact, torque, moment of inertia, centripetal force, work, power, kinetic energy, potential energy, angular momentum, and centripetal acceleration.

As an example, the controller 700 calculates the dynamical element of each joint over time upon exercise, based on the foot pressure applied to the feet of the user doing exercise using the barbell (S3380).

Then, the controller 700 identifies (or recognizes) the free weight training motion which the user is doing using the piece of exercise equipment, based on, e.g., the identified joint motion information (or body joint information), exercise type and number of repetitions of exercise, the identified kind and weight of piece of exercise equipment, the identified foot position, the weight distribution of foot, pressure distribution of foot, and per-foot section pressures (or per-foot section pressure distribution).

As an example, the controller 700 identifies the barbell shoulder press of free weight training motion, which the user is doing using the barbell, based on the joint motion information (or body joint information) identified via the first image information and/or second image information, type and repeat count of exercise, identified kind of piece of exercise equipment (e.g., barbell) and weight (e.g., 5 kg), the identified position of feet upon exercise, and per-foot section pressure distribution (S3390).

Thereafter, the controller 700 compares standard posture information as per exercise types preset per piece of exercise equipment, pre-stored in the storage unit 400, with the identified user body information, joint motion information (or body joint information), exercise type and weight of piece of exercise equipment as per the number of repetitions of exercise, foot position, weight distribution of foot, foot pressure distribution, and per-foot section pressure distribution. The standard posture information includes body information corresponding to the optimal exercise course and an athlete with superior performance (e.g., a gold medalist in an international game), joint motion information related to the free weight training motion corresponding to the athlete, the number of repetitions as per the weight and exercise type of piece of exercise equipment, foot position, foot weight distribution, foot pressure distribution, and per-foot section pressure distribution.

The controller 700 compares standard posture information as per exercise types preset per piece of exercise equipment, pre-stored in the storage unit 400, with the identified user body information, joint exercise information, body and/or joint position information, per-section time information of motion upon exercise, foot pressure distribution, and per-foot section pressure distribution.

As an example, the controller 700 compares the standard posture information per piece of exercise equipment, pre-stored in the storage unit 400, with the identified user body information and information related to the barbell shoulder press being done by the user (S3400).

Thereafter, the controller 700 generates feedback information related to the user's free weight training motion according to the result of comparison. The feedback information includes, e.g., the optimal exercise course information which is information customized to the user, information about whether the user's exercise goal is achieved or not, the dynamical element of each joint (or dynamical element of body) according to the user's exercise goal, information for partial reinforcing exercise, information for retraining exercise technique (including, e.g., information about the body and/or joint motion or posture, foot position information, foot weight distribution information, foot pressure distribution information, and per-foot section pressure distribution), comparison image information for standard posture information and the foot position and the motion of each joint and/or body of the captured user, user recognition result (or user information), and user's body information. The optimal exercise course information includes, e.g., body posture information according to a performing of the exercise, using the obtained gravity line (or center line) and body median line of the user per exercise section over time. The information about whether the user's exercise goal is achieved includes information about whether the user has normally met the target according to the exercise goal according to the result of comparison and the exercise performing rate according to the exercise goal. The exercise goal may target reinforcing muscle strength, explosive power, muscle hypertrophy, and muscle endurance.

Further, the controller 700 provides (or outputs) the generated feedback information to the user.

As an example, the controller 700 displays, via the display unit 500, information for comparison between the user's mid-exercise posture and the standard posture information in relation to the barbell shoulder press being done by the user according to the result of comparison, and information for comparison between the user's body information and the body information of a gold medalist related to the standard posture information in relation to the barbell shoulder press being done by the user, allowing the user to easily identify the information, which needs to be modified, while doing the barbell shoulder press (S3410).

According to an embodiment, as described above, various pieces of information, such as motion for, e.g., the phase, angle, speed, displacement, and depth of major body joints of the user doing exercise using the piece of exercise equipment, exercise type, exercise repeat count, and exercise weight may be recognized using the depth camera or various sensors, and the free weight training motion which the user is doing on the piece of exercise equipment may be recognized based on the recognized pieces of information. Thus, the free weight training motion being done by the user may be identified easily and conveniently.

Further, according to an embodiment of the disclosure, as described above, feedback for the user's body information, reeducation of exercise technique, and partial reinforcing exercise may be provided via comparison between pre-stored pieces of information related to the free weight training motion corresponding to an athlete with superior performance and the optimal exercise course with pieces of information gathered in relation to the free weight training motion the user is doing using the piece of exercise equipment. Thus, the user may be given the optimized customized exercise type, and a desired part may be specifically reinforced.

The above-described embodiments are merely examples, and it will be appreciated by one of ordinary skill in the art various changes may be made thereto without departing from the scope of the present disclosure. Accordingly, the embodiments set forth herein are provided for illustrative purposes, but not to limit the scope of the present disclosure, and should be appreciated that the scope of the present disclosure is not limited by the embodiments. The scope of the present disclosure should be construed by the following claims, and all technical spirits within equivalents thereof should be interpreted to belong to the scope of the present disclosure.

What is claimed is:

1. A device for recognizing a free weight training motion, comprising:
a depth camera obtaining image information for measuring a user's body information before exercise and obtaining image information including a unique identifier attached to a piece of exercise equipment and motion of each joint of the user and a variation in body surface due to a motion of the body of the user doing exercise using the piece of exercise equipment;
a sensor unit measuring a state of the piece of exercise equipment and recognizing the user; and
a controller identifying motion information of a joint, on which the user is doing exercise using the piece of exercise equipment, type and repeat count of exercise, the user's body information, kind and weight of the piece of exercise equipment, and foot position and per-foot section pressure distribution, over time, upon exercise, based on the obtained image information and the measured state of piece of exercise equipment, identifying the free weight training motion being done by the user using the piece of exercise equipment, based on the identified joint motion information, exercise type and repeat count, the identified kind and weight of the piece of exercise equipment, and the identified foot position and per-foot section pressure distribution, comparing standard posture information according to exercise types pre-configured per piece of exercise equipment, previously stored in a storage unit, with the identified user body information, joint motion information, weight of the piece of exercise equipment according to the exercise type and repeat count, and foot position and per-foot section pressure distribution, and generating feedback information related to the user's free weight training motion according to a result of the comparison.

2. The device for recognizing a free weight training motion of claim 1, wherein
the sensor portion includes:
an ultrasonic sensor measuring a motion range of section in which the piece of exercise equipment is repeatedly moved;
an radio frequency identification (RFID) reader recognizing the unique identifier attached to the piece of exercise equipment;
a hall sensor measuring a magnitude of a magnetic field for a magnet attached to the piece of exercise equipment;
a magnetic sensor measuring a magnitude of a magnetic field for a magnet attached to the piece of exercise equipment;
a pressure plate measuring the user's body motion and foot pressure per exercise section for the motion of each joint, over time, for the user doing exercise using the piece of exercise equipment;
a face recognition sensor recognizing the user doing exercise using the piece of exercise equipment;
a fingerprint recognition sensor recognizing the user doing exercise using the piece of exercise equipment; and
an iris recognition sensor recognizing the user doing exercise using the piece of exercise equipment.

3. The device for recognizing a free weight training motion of claim 1, wherein
the controller
identifies the type and repeat count of exercise being done by the user using the piece of exercise equipment, based on at least one of the motion of each joint of the user in the obtained image information and the motion range of section, in which the piece of exercise equipment is repeatedly moved, the motion range of section being included in the measured state of the piece of exercise equipment,
identifies the kind and weight of the piece of exercise equipment corresponding to the unique identifier, based on at least one of the unique identifier attached to the piece of exercise equipment in the obtained image information and a unique identifier recognized from the piece of exercise equipment included in the measured state of the piece of exercise equipment, and
identifies at least one of the foot position and per-foot section pressure distribution, over time, upon exercise, based on the per-exercise section foot pressure for the motion of the body and each joint, over time, included in the measured state of the piece of exercise equipment.

4. A method for recognizing a free weight training motion, comprising:
obtaining, by a depth camera, image information for measuring a user's body information before exercise and image information including a unique identifier attached to a piece of exercise equipment and motion of each joint of the user and a variation in body surface due to a motion of the body of the user doing exercise using the piece of exercise equipment;
measuring, by a sensor unit, a state of the piece of exercise equipment and recognizing the user;
identifying, by a controller, joint motion information, type, and repeat count of exercise being done by the user using the piece of exercise equipment, based on at least one of the motion of each joint of the user in the obtained image information and the motion range of section, in which the piece of exercise equipment is repeatedly moved, the motion range of section being included in the measured state of the piece of exercise equipment;
identifying, by the controller, the kind and weight of the piece of exercise equipment corresponding to the unique identifier, based on at least one of the unique identifier attached to the piece of exercise equipment in the obtained image information and a unique identifier recognized from the piece of exercise equipment included in the measured state of the piece of exercise equipment;
identifying, by the controller, at least one of the foot position and per-foot section pressure distribution, over time, upon exercise, based on the per-exercise section foot pressure for the motion of the body and each joint, over time, included in the measured state of the piece of exercise equipment;
identifying, by the controller, a free weight training motion being done by the user using the piece of exercise equipment, based on the identified joint motion information, exercise type and repeat count, the identified kind and weight of the piece of exercise equipment, and the identified foot position and per-foot section pressure distribution;
comparing, by the controller, standard posture information according to exercise types pre-configured per piece of exercise equipment, previously stored in a storage unit, with the identified user body information, joint motion information, weight of the piece of exercise equipment according to the exercise type and repeat count, and foot position and per-foot section pressure distribution;
generating, by the controller, feedback information related to the user's free weight training motion according to a result of the comparison; and
providing, by the controller, the generated feedback information to the user.

5. The method for recognizing a free weight training motion of claim 4, wherein
the feedback information includes at least one of optimal exercise course information customized to the user, information as to whether the user's exercise goal is achieved, a dynamical element of each joint according to the user's exercise goal, information for partial strengthening exercise, information for reeducation of an exercise technique, comparison image information for the standard posture information and the captured motion of the user's body and each joint or foot position, a result of user recognition, and the user's body information.
